(19) United States
(12) Patent Application Publication
(10) Pub. No.: US 2021/0068400 A1
(43) Pub. Date: Mar. 11, 2021

(54) COMPOSITIONS CONTAINING COMBINATIONS OF NITROGEN-FIXING BACTERIA AND ADDITIONAL AGENTS AND THEIR USE IN FIXING NITROGEN IN PLANT SPECIES

(71) Applicant: GRACE BREEDING NITROGEN FIXATION TECHNOLOGIES LTD., Kfar Saba (IL)

(72) Inventors: Amit AVIDOV, Kiryat Tivon (IL); Avner BARAZANI, Omer (IL); Mor ZEILKHA, Ramat Gan (IL)

(21) Appl. No.: 16/963,674

(22) PCT Filed: Jan. 21, 2019
(86) PCT No.: PCT/IL2019/050082

§ 371 Date: Jul. 21, 2020

Related U.S. Application Data
(60) Provisional application No. 62/619,848, filed on Jan. 21, 2018.

(51) Int. Cl. A01N 63/20; A01N 31/06; A01N 43/16; A01N 35/06; A01N 31/16; A01N 65/12; A01N 65/40; C05B 17/00; A01N 25/12

(52) U.S. Cl. CPC A01N 63/20; A01N 31/06; A01N 43/16; A01N 35/06; A01N 25/12; A01N 65/12; A01N 65/40; C05B 17/00; A01N 31/16

(57) ABSTRACT

The present invention provides a method for supplying the nitrogen requirements of a plant comprising administering to said plant a combination of non-pathogenic, atmospheric nitrogen-fixing bacteria and one or more activating agents. Many of these activating agents possess potent anti-inflammatory and anti-microbial activity. The method is particularly suitable for use in enabling nitrogen fixation in plant species such as wheat, maize and other cereal crops, in which nitrogen fixation is normally not possible. The invention also provides compositions comprising nitrogen-fixing bacteria and suitable activating agents. In one preferred embodiment, the nitrogen fixing bacteria are of the Rhizobium genus.

### FIELD OF THE INVENTION

The present invention relates to compositions and methods for enhancing nitrogen fixation in plants. More specifically, the invention provides combinations of non-pathogenic, atmospheric nitrogen fixing, bacteria with one or more additional agents, and the use of said combinations in the fixation of nitrogen in several different species including graminaceous plants.

### BACKGROUND OF THE INVENTION

Nitrogen fixation is a process by which nitrogen in the Earth's atmosphere is converted into ammonia or other nitrogen-containing molecules which are then made available to living organisms for their metabolic and biosynthetic needs. In the case of plants, supply of nitrogen is needed from the early stage following germination until the plant has matured and developed its full crop yield potential.

The Gramineae family includes maize wheat and rice, which are the three main crops used world-wide for feeding the human population.

Unlike the Leguminosae plants that can fix atmospheric nitrogen by symbiosis with certain bacterial species, including those of the Rhizobium genus, the Gramineae family is not able to fix atmospheric nitrogen and growers need to use chemical fertilizers to supply the plants with the required amount of nitrogen, in order to improve crop yields.

This method of chemical fertilization, however, is not without significant problems, not least of which is massive contamination of the fresh water resources on the planet, leading to severe ecological damage. This may occur, for example, when nitrogen-containing fertilizers are washed out from the root zone of the plants and leak into the deeper aquifers and the fresh water reservoirs.

An urgent need therefore exists for alternative methods and compositions for enabling and/or enhancing the fixation of nitrogen in many plant species, in particular those of the Gramineae family. The present invention provides a solution for this need.

### SUMMARY OF THE INVENTION

The present inventors have unexpectedly found that when certain bacteria, such as Rhizobium species are administered to plants in combination with certain other substances (as will be disclosed and described in detail hereinbelow), said combinations are capable of fixing atmospheric nitrogen and thereby supply the plant's nitrogen needs. This effect is particularly unexpected when the plants so treated are those of the Gramineae family, which, as explained hereinabove, are normally unable to obtain their nitrogen requirements by means of nitrogen-fixation mediated by bacteria present in the soil.

The present invention is primarily directed to a method for completely or partially supplying the nitrogen requirements of a plant, by means of administering to said plant a combination of non-pathogenic atmospheric nitrogen fixing bacteria together with one or more activating agents. In some cases, one or more fertilizers is also supplied together with said bacteria and activating agents. Thus, the present invention is primarily directed to a method for enabling fixation of atmospheric nitrogen in plant species that are normally unable to obtain their nitrogen intake in this manner.

In another aspect, the present invention provides a composition comprising a mixture of non-pathogenic nitrogen-fixing bacteria and one or more activating agents (as defined hereinabove and described hereinbelow).

In a further aspect, the present invention also provides a method for increasing the yield of a plant of agricultural or horticultural importance by means of:
a) providing a composition comprising a combination of a non-pathogenic nitrogen-fixing bacteria and one or more activating agents as disclosed hereinbelow; and
b) administering the composition of step (a) to said host species.

In a still further aspect, the present invention further provides a method for increasing the yield of a plant of agricultural or horticultural importance by means of:

a) providing separately:
(i) a composition comprising one or more nitrogen fixing non-pathogenic bacteria; and
(ii) a composition comprising one or more activating agents as disclosed and defined hereinbelow; and

b) separately administering each of compositions (i) and (ii) to said host species.

In the above-disclosed methods and compositions, the nitrogen fixing non-pathogenic bacteria are, in one embodiment, members of the Rhizobium genus. In one preferred embodiment, the bacteria are of the species Rhizobium leguminosarum. Further examples of suitable bacteria will be disclosed hereinbelow.

In the above-disclosed methods the plant of agricultural or horticultural importance is, in one embodiment, a member of a species which is normally unable to obtain its nitrogen requirements by bacterial fixation of atmospheric nitrogen. In one embodiment, said plant species is a member of the Graminaea family. In one preferred embodiment, the plant species is maize. In another preferred embodiment, the plant species is wheat. In a still further preferred embodiment, the plant species is rice.

### BRIEF DESCRIPTION OF THE DRAWINGS

FIG. 1 graphically presents the nitrogen content in the leaves of maize plants treated with a composition of the present invention.

FIGS. 2A and 2B presents results showing increased maize plant height following treatment with a composition of the present invention.

FIGS. 3A and 3B presents results for the percentage of maize plants that show signs of silking following treatment with a composition of the present invention.

FIGS. 4A and 4B graphically show the effect of treatment with a composition of the present invention on male flower formation in maize plants.

FIG. 5 presents results for cob formation in maize plants treated with a composition of the present invention

FIGS. 6A and 6B present results comparing the degree of green color of the foliage in treated and untreated maize plants.

FIG. 7 graphically depicts the increase in nitrogen content of maize plants treated with a composition of the present invention.

FIGS. 8A and 8B present data showing the effect of the treatment of the present invention on the height of maize plants.

FIGS. 9A and 9B present results of the effect of a composition of the invention on the amount of silking seen in maize plants.

FIGS. 10A and 10B present results showing the effect of a composition of the invention on male flower formation in maize plants.

FIG. 11 graphically presents data summarizing the effect of composition of the present invention on cob formation in treated maize plants.

FIGS. 12A and 12B present data showing the difference in the degree of green coloration between treated and untreated maize plants.

FIG. 13 is a comparative photograph showing the difference in green coloration and general vitality of treated and untreated maize plants.

FIG. 14 compares the mean plant stem thickness of treated and untreated maize plants.

FIG. 15 compares the mean leaf width of treated and untreated maize plants.

FIG. 16 compares the mean cob weight obtained from treated and untreated maize plants.

FIG. 17 compares the mean total plant weight of treated and untreated maize plants.

FIG. 18 compares the mean plant height of treated and untreated maize plants.

FIG. 19 presents results for average total nitrogen content of leaves taken from treated and untreated maize plants.

FIG. 20 is a photographic representation of the root of an untreated wheat plant.

FIG. 21 is a photographic representation of the root of a wheat plant treated with a composition of the present invention.

FIG. 22 is a photographic representation of the root of a wheat plant treated with a different dosage of a composition of the present invention.

FIG. 23 graphically presents data for (from left to right): main shoot diameter, flag leaf width and the number of side shoots, in wheat.

FIG. 24 summarizes the flag leaf nitrogen content data for wheat treated with a composition of the present invention.

FIG. 25 summarizes the average wheat grain yield for wheat treated with a composition of the present invention.

FIG. 26 presents in vitro results obtained for the effect of compositions of the present invention on fungal elimination, bacterial elimination and Rhizobium species activation

FIGS. 27A, 27B and 27C summarize in vitro results for the fungal, bacterial and activation indices.

FIGS. 28A, 28B and 28C summarize in vitro results for the fungal, bacterial and activation indices, using a different concentration of activating agents.

FIG. 29 presents in vitro results for the fungal, bacterial and activation indices, using a different Rhizobium composition.

FIG. 30 presents in vitro results for the fungal, bacterial and activation indices, using a different concentration of activating agents from that employed in FIG. 29.

FIG. 31 present the results of an inoculation study in tomato plants.

FIG. 32 present the results of an inoculation study in cucumber seedlings.

### DETAILED DESCRIPTION OF THE EMBODIMENTS

The present inventor have found, as disclosed hereinabove, that certain combinations of non-pathogenic nitrogen fixing bacteria and activating factors (whose properties will be described in detail hereinbelow) are capable of permitting nitrogen fixation in plant species (such as cereals) which are normally unable to obtain their nitrogen requirements in this way.

It has also been found by the inventors that the same combinations of nitrogen-fixing bacteria and activating factors also possess both anti-inflammatory and anti-microbial properties (directed against several different bacterial and fungal species, including those known to be plant pathogens).

The reason for this correlation between the ability of these combinations to permit nitrogen fixation in species that are normally unable to obtain nitrogen in this way and their anti-inflammatory and anti-microbial properties is not entirely clear.

Without wishing to be bound by theory, it is believed that by means of administering bacteria of the Rhizobium genus with the additional substances and agents set out in this disclosure, symbiosis develops between said bacteria and the root systems of plants of species such as those of the Gramineae family, thereby enabling fixation of atmospheric nitrogen within the plant. Again, without being bound by theory, it is possible that the reason that this symbiosis does not occur in the absence of said additional agents may be rejection of the Rhizobium bacteria by the Gramineae plants. It is therefore possible that the additional substances and agents which permit the aforementioned symbiosis to take place do so by means of preventing the development of this rejection mechanism.

Thus, by these means, plants of the Gramineae family—and of other species which are similarly unable to obtain their nitrogen needs via nitrogen-fixing bacteria alone—are able to satisfy their nitrogen requirements.

In one preferred embodiment, the plant is a species which is normally unable to obtain its nitrogen requirements by bacterial fixation of atmospheric nitrogen. In one particularly preferred embodiment, the plant species is a member of the Gramineae family. One example of such a species is maize (Zea mays)

In one preferred embodiment, the non-pathogenic atmospheric nitrogen fixing bacteria are bacteria belonging to the general class known as Rhizobia. The bacteria of this class are distributed among several different genii, and have the common feature of being able to fix nitrogen in certain plant species (such as legumes), after having been established within the root nodules of said plants.

Thus, in one embodiment of the present invention, the non-pathogenic atmospheric nitrogen fixing bacteria are Rhizobia, belonging to one or more genii selected from the group consisting of Bosea, Ochrobactrum, Devosia, Methylobacterium, Phyllobacterium, Rhizobium, Shinella, Sinorhizobium/Ensifer, Azorhizobium, Burkholderia and Cupriavidus.

In one particularly preferred embodiment, the Rhizobial bacteria are of the Rhizobium genus. Many different species of Rhizobium may be used in the combinations of the present invention, including R. alamii, R. alkalisoli, R. cauense, R. cellulosilyticum, R. daejeonense, R. etli, R. fabae, R. galegae, R. gallicum, R. giardinii, R. grahamii, R. hainanense, R. halophytocola, R. helanshanense, R. herbae, R. huautlense, R. indigoferae, R. leguminosarum, R. leucaenae, R. loessense, R. lupini, R. lusitanum, R. mesoamericanum, R. mesosinicum, R. miluonense, R. mongolense, R. multihospitium, R. nepotum, R. oryzae, R. petrolearium, R. phaseoli, R. pisi, R. pusense, R. qilianshanense, R. sphaerophysae, R. sullae, R. taibaishanense, R. tibeticum, R. tropici R. tubonense, R. undicola, R. vallis, R. vignae and R. yanglingense.

In some other embodiments, the nitrogen-fixing bacteria used to work the present invention may be of the Bradyrhizobim genus, for example, a species such as Bradyrhizobium japonicum.

In some cases, the Rhizobium species selected may be one which is already in commercial use for providing nitrogen requirements of leguminous species such as peanuts (groundnuts) and soya.

However, in one particularly preferred embodiment, the species used is Rhizobium leguminosarum. Although several different biovars of this species exist, in one preferred embodiment of the present invention, the biovar used is R. leguminosarum biovar viceae.

In another preferred embodiment, the non-pathogenic atmospheric nitrogen fixing bacteria are bacteria of the Clostridium genus. These anaerobic bacteria are particularly preferred when the combinations of the present invention are administered to crops such as rice which are grown under water-logged conditions. In one preferred embodiment of this aspect of the invention, the nitrogen-fixing Clostridium are selected from the group consisting of C. pasteurianum, C. acetobutylicum, C. beijerinckii, C. butyicum, C. hungatei and C. acidisoli.

It is to be noted that the term “nitrogen fixing bacteria” is used to indicate that these bacteria are generally capable of fixing atmospheric nitrogen in a large variety of vegetable and legume species, many of which (such as soya and peanuts) are of great economic value. However, as noted hereinabove, these bacteria, by themselves, are incapable of causing nitrogen fixation in cereal crops and rice.

In the context of the present invention, the term “activating agent” is used to denote a substance which when present in a mixture together with the non-pathogenic nitrogen fixing bacteria or when delivered separately therefrom, enables fixation of atmospheric nitrogen when administered to growing plant species that are normally unable to obtain their nitrogen requirements by this route. In some cases, this effect may be seen to be the result of a synergistic interaction between the non-pathogenic nitrogen fixing bacteria and the activating agents.

The present inventors have unexpectedly found that many of the activating agents suitable for use in the method of the present invention share a common feature, namely their ability to inhibit inflammatory mediators that are more generally associated with higher animal species (such as Tumor Necrosis Factor alpha [TNF-α]) rather than with plant species. Thus, in one preferred embodiment of the present invention, the one or more activating agents are substances having anti-inflammatory activity.

In one embodiment of the method of the invention, activating agents each have an IC₅₀ for the inhibition of NO production of less than 1.6 mg/ml and/or an IC₅₀ for the inhibition of TNF-α production of less than 2.4 mg/ml.

In another preferred embodiment of the method, each individual activating agents (whether used alone or in combination with other such agents) has an IC₅₀ for the inhibition of NO production of less than 0.4 mg/ml and/or an IC₅₀ for the inhibition of TNF-α production of less than 2.4 mg/ml.

In another preferred embodiment of the method, each individual activating agents (whether used alone or in combination with other such agents) has an IC₅₀ for the inhibition of NO production of equal to or less than 0.15 mg/ml and/or an IC₅₀ for the inhibition of TNF-α production of equal to or less than 2.4 mg/ml.

In another preferred embodiment of the method, each individual activating agents (whether used alone or in combination with other such agents) has an IC₅₀ for the inhibition of NO production of equal to or less than 0.1 mg/ml and/or an IC₅₀ for the inhibition of TNF-α production of equal to or less than 0.2 mg/ml.

In a still further preferred embodiment of the method, each individual activating agents (whether used alone or in combination with other such agents) has an IC₅₀ for the inhibition of NO production of equal to or less than 0.05 mg/ml and/or an IC₅₀ for the inhibition of TNF-α production equal to or less than 0.1 mg/ml.

In another preferred embodiment of the method, the activating agents are selected from the group consisting of Sclareol, Naringin, Nootkatone, Steviol glycoside and cannabidiol (CBD) and combinations thereof.

In one particularly preferred embodiment of the method, the one or more activating agents comprises cannabidiol (CBD). In this embodiment, the activating agents used in the method may further comprise agents or substances each having an IC₅₀ for the inhibition of NO production of less than 1.6 mg/ml and/or an IC₅₀ for the inhibition of TNF-α production of less than 2.4 mg/ml.

Said CBD may be obtained from many different sources, but in one preferred embodiment is supplied in the form of hemp oil.

In a yet further preferred embodiment of the method, the activating agents (including those having the qualitative and quantitative anti-inflammatory properties disclosed above) are derived from plant material (such as crude plant extracts, such as whole plant aqueous extracts, partially purified or fractionated extracts, purified extracts and synthetic analogues of active molecules present in said extracts).

In one preferred embodiment of this aspect of the invention, the plant-derived activating agents are herbal extracts selected from the group consisting of Aster tataricus, Cyperus rotundus and combinations thereof.

While the method of the present invention may be employed to promote nitrogen fixation in almost any vegetable or legume plant of commercial importance, in one preferred embodiment, the plant treated in the present method is a member of a species which is normally unable to obtain its nitrogen requirements by bacterial fixation of atmospheric nitrogen. In one preferred embodiment, the plant species is a member of the Graminaea family. Preferred (but non-limiting) examples of such species include maize, wheat and rice. In one particularly preferred embodiment, the plant species is maize. In another, it is wheat.

In some embodiments, the method of the present invention may further comprise the administration phosphorous-containing fertilizers. In one preferred embodiment, the fertilizer is Calirus.

In some embodiments of the presently-disclosed method, the combination of non-pathogenic, atmospheric nitrogen-fixing bacteria and one or more activating agents are administered by means selected from the group consisting of: application of slow-release granules to the soil in which the plants are being grown, seed coating and spraying the sowing trench or furrow. In some cases, the non-pathogenic, atmospheric nitrogen-fixing bacteria and the one or more activating agents are administered together in a single composition. In other embodiments, however, the non-pathogenic, atmospheric nitrogen-fixing bacteria and the one or more activating agents are administered in separate compositions.

In another aspect, the present invention provides a composition comprising a mixture of non-pathogenic nitrogen-fixing bacteria and one or more activating agents (as defined hereinabove and described hereinbelow).

Many different species and strains of non-pathogenic nitrogen-fixing bacteria may be used in combination with the activating agents described herein (i.e. in a single composition), or alternatively, may be administered in separate compositions. In the latter case, the two or more compositions may be administered either simultaneously or sequentially. The term ‘non-pathogenic’ is used in this context to indicate that the selected species have no, or very few, toxic or other deleterious effects on the host species to which the composition of the invention containing the bacteria are being administered.

In one preferred embodiment of the methods and compositions defined herein, the non-pathogenic bacteria are of the Rhizobia class. Suitable genii and species are disclosed herein.

In another preferred embodiment, the non-pathogenic nitrogen-fixing bacteria are of the Clostridium genus, in particular those species that are disclosed herein.

In some preferred embodiments the composition of the present invention further comprises (in addition to the non-pathogenic nitrogen fixing bacteria and the one or more activating agents) one or more phosphorous-containing fertilizers. Suitable fertilizers for this purpose include (but are not limited to) commercially-available preparations such as Calirus.

In one preferred embodiment, the combination of non-pathogenic bacteria, activating agents and fertilizers (when present) may be administered as a single composition. In other embodiments, some of these components may be administered separately.

Routes of administration of the combinations of the present invention include (but are not limited to) application of slow-release granules to the soil in which the plants are being grown, seed coating and spraying the sowing trench or furrow.

As mentioned hereinabove, it has been found by the present inventors that in some embodiments, the activating agents of the present invention may be characterized by their ability to inhibit one or more key inflammatory mediators such as TNF-α and/or nitric oxide (NO). Consequently, in one preferred embodiment of the present invention, the one or more activating agents used in the aforementioned method are substances capable of inhibiting the production of NO and/or TNF-α.

In one further preferred embodiment of the present invention, the activating agents each have an IC₅₀ for the inhibition of NO production of less than 1.6 mg/ml and/or an IC₅₀ for the inhibition of TNF-α production of less than 2.4 mg/ml.

In another preferred embodiment, each individual activating agents (whether used alone or in combination with other such agents) has an IC₅₀ for the inhibition of NO production of less than 0.4 mg/ml and/or an IC₅₀ for the inhibition of TNF-α production of less than 2.4 mg/ml.

In another preferred embodiment, each individual activating agents (whether used alone or in combination with other such agents) has an IC₅₀ for the inhibition of NO production of equal to or less than 0.15 mg/ml and/or an IC₅₀ for the inhibition of TNF-α production of equal to or less than 2.4 mg/ml.

In another preferred embodiment, each individual activating agents (whether used alone or in combination with other such agents) has an IC₅₀ for the inhibition of NO production of equal to or less than 0.1 mg/ml and/or an IC₅₀ for the inhibition of TNF-α production of equal to or less than 0.2 mg/ml.

In a still further preferred embodiment, each individual activating agents (whether used alone or in combination with other such agents) has an IC₅₀ for the inhibition of NO production of equal to or less than 0.05 mg/ml and/or an IC₅₀ for the inhibition of TNF-α production equal to or less than 0.1 mg/ml.

It is to be noted that the use of the IC₅₀ value (i.e. the concentration of an agent which causes 50% of the maximal inhibition of a mediator, agonist or other biologically active molecule) as a means for comparing the potency of antagonists and other biologically- and pharmacologically-active molecules, is well-known to all skilled-artisans in this field. Briefly, the IC₅₀ values may be obtained by plotting dose-response curves for a parameter such as inhibition of a particular inflammatory mediator, and extracting said values from said curves.

In another preferred embodiment, the activating agents are selected from the group consisting of Sclareol, Naringin, Nootkatone, Steviol glycoside and cannabidiol (CBD) and combinations thereof.

In one particularly preferred embodiment, the one or more activating agents comprises cannabidiol (CBD). In this embodiment, the activating agents used in the method may further comprise agents or substances each having an IC₅₀ for the inhibition of NO production of less than 1.6 mg/ml and/or an IC₅₀ for the inhibition of TNF-α production of less than 2.4 mg/ml.

Said CBD may be obtained from many different sources, but in one preferred embodiment is supplied in the form of hemp oil.

In a yet further preferred embodiment, the activating agents (including those having the qualitative and quantitative anti-inflammatory properties disclosed above) are derived from plant material (such as crude plant extracts, such as whole plant aqueous extracts, partially purified or fractionated extracts, purified extracts and synthetic analogues of active molecules present in said extracts).

In one preferred embodiment of this aspect of the invention, the plant-derived activating agents are herbal extracts selected from the group consisting of Aster tataricus, Cyperus rotundus and combinations thereof. Further suitable plant extracts are disclosed elsewhere herein.

In another aspect, the present invention also provides a method for increasing the yield of a plant of agricultural or horticultural importance by means of:
a) providing a composition comprising a combination of a non-pathogenic nitrogen-fixing bacteria and one or more activating agents as disclosed herein; and
b) administering the composition of step (a) to said host species.

The present invention further provides a method for increasing the yield of a plant of agricultural or horticultural importance by means of:

a) providing separately:
(i) a composition comprising one or more nitrogen fixing non-pathogenic bacteria; and
(ii) a composition comprising one or more activating agents as disclosed and defined herein; and

b) separately administering each of compositions (i) and (ii) to said host species.

In the above-disclosed methods, the nitrogen fixing non-pathogenic bacteria are, in one embodiment, members of the Rhizobium genus. In one preferred embodiment, the bacteria are of the species Rhizobium leguminosarum.

In the above-disclosed methods the plant of agricultural or horticultural importance is, in one embodiment, a member of a species which is normally unable to obtain its nitrogen requirements by bacterial fixation of atmospheric nitrogen. In one embodiment, said plant species is a member of the Graminaea family. In one preferred embodiment, the plant species is maize. In another preferred embodiment, the plant species is wheat. In a still further preferred embodiment, the plant species is rice.

The advantages and benefits of the present invention will now be described in more detail in the following working Examples and accompanying drawings.

### EXAMPLES

### General Methods

### 1. Preparation of the Activating Agent Emulsion

In this study, the following activating agents were mixed together and used in combination with the nitrogen fixing bacteria:

Sclareol, nootkatone, cannabidiol (CBD), naringin, steviol.

Since naringin and steviol are water soluble, while the other three activating agents are lipid soluble, two separate solutions—an oil phase and an aqueous phase—were prepared, as summarized in the following table, and then mixed using a high-shear mixer. As will be seen from this table, the oil phase contained (in addition to three of the activating agents) medium chain triglycerides (MCT) and a hydrolyzed sunflower lecithin (Giralec HE-60; E-322), while the aqueous phase also comprises water, glycerol and the non-ionic surfactant, sucrose palmitate (Sisterna PS750):

| | | per 1 Liter, | weight, | | |
|---|---|---|---|---|---|
| | ingrediants | g | g | % | 200 |
| Oil phase | Sclareol | 8.00 | | 0.8%% | 1.6 |
| Oil phase | Nootkatone | 8.00 | | 0.8%% | 1.6 |
| Oil phase | CBD | 8.00 | | 0.8%% | 1.6 |
| Oil phase | Giralec | 50.00 | 50 | 5.00% | 10 |
| | HE-60 | | | | |
| | (lecitin | | | | |
| Oil phase | MCT | 80.00 | 80 | 8.00% | 16 |
| Oil phase | | | | | |
| Oil phase | sum oil | 154.00 | | 15.40% | 30.8 |
| | phase | | | | |
| Water phase | PS 750 | 22.00 | 20 | 2.20% | 4.4 |
| Water phase | Naringin | 8.00 | | 0.80% | 1.5 |
| Water phase | Steviol | 14.00 | | 1.40% | 2.8 |
| Water phase | water | | | 20.00% | 40 |
| Water phase | glycerol | | | 60.20% | 120.4 |
| | | | | 24.40% | |
| Emulsion | | 1000 | | 100.00% | |
| Active | | 4.60% | | | |
| Ingr . . . % | | | | | |

The drop size of the emulsion following mixing in the high-shear mixer was 214 nm.

### 2. Preparation of the Treatment Granules

In some of the treatments (as explained hereinbelow), some or all of the active substances were added in the form of granules to the furrow in which the plants had been seeded. The granules were prepared by soaking 1 kg of Perlite granules (having a mean diameter greater than 2 mm) in the following solution:
50 ml of the concentrated activating agent emulsion (as described above).
930 ml water.
10 ml Rhizobium leguminosarum biovar viceae (Cell-Tech™ granular; Monsanto Company) (Granule-R). or Bacillus subtilis (Granule-B)
10 ml Calirus.
1000 ml total

### 3. Measurement of Nitrogen Content of Plant Material

Generally, the nitrogen content of the growing plant in the field study was measured using a spectrophotometric method based on the standard method “4500-NO3_I. Cadmium Reduction Flow Injection Method” published by the Standard Methods Organization (<https://www.standardmethods.org/doi/full/10.2105/SMWW.2882.089). This method is based on the conversion of nitrates in an aqueous plant material extract to nitrites by passing the extract through a copperized cadmium column. Subsequent processing steps convert the nitrites to a magenta-colored dye having an absorbance peak at 540 nm.

### Example 1

### Treatment of Growing Maize Plants with a Composition of the Present Invention: Field Trial 1

### Method

The trial was sown on the format date 29 of Aug. 2017 using the pioneer maize silage variety number 32-W-68. The field was washed from possible nitrogen using sprinklers irrigation. The combination treatments administered to the plants which are of relevance for the present study were:
C=Untreated Control without nitrogen.
F=Rhizobium leguminosarum biovar viceae, 1% emulsion diluted by 20 and Calirus 1% sprayed in the sowing trench.
H=Rhizobium leguminosarum biovar viceae, 1% emulsion diluted by 20 and Calirus 1% sprayed in the sowing trench+granules containing the emulsion and Bacillus s. 0.5% (granule-B).

The amount of the solution containing the Rhibozium 1%, the activating agent emulsion and the fertilizer (Calirus) (treatments F and H) was calculated such that 2 liters per 1000 m row were added to the sowing trench.

In the case of treatment H, the granule quantity was adjusted to 4 Kg granules per 1000 m².

The following parameters were monitored at either one timepoint (Oct. 29, 2017) or two timepoints (October 23 and Oct. 29, 2017) during growth of the maize plants:
Nitrogen % in the leaves
Plant height
Flowering (silking and male flowering)
Second cob %
Foliage color

### Results

### 1. Nitrogen Content in the Leaves

| | C | H | F |
|---|---|---|---|
| Average | 1.88 | 3.72 | 3.11 |
| S.D. | 0.22 | 1.32 | 0.34 |

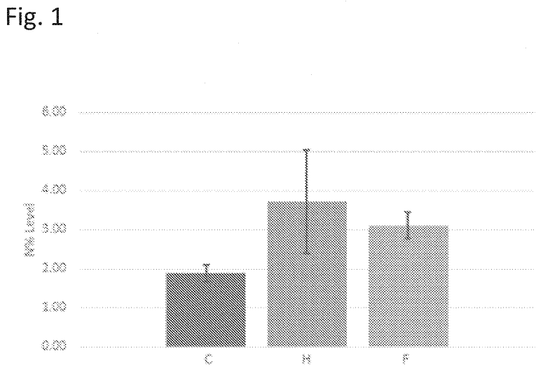

As may be seen from the table and from the graph shown in FIG. 1, treatments H and F result in significantly higher nitrogen levels, compared to the untreated control. These results indicate that these treatments enable the plants to absorb and fix nitrogen from the atmosphere.

### 2. Plant Height

| | | C | H | F |
|---|---|---|---|---|
| 23 Oct. 2017 | Average | 1.18 | 1.30 | 1.50 |
| | SD | 0.10 | 0.08 | 0.08 |
| 29 Oct. 2017 | Average | 1.45 | 1.70 | 1.70 |
| | SD | 0.13 | 0.14 | 0.08 |

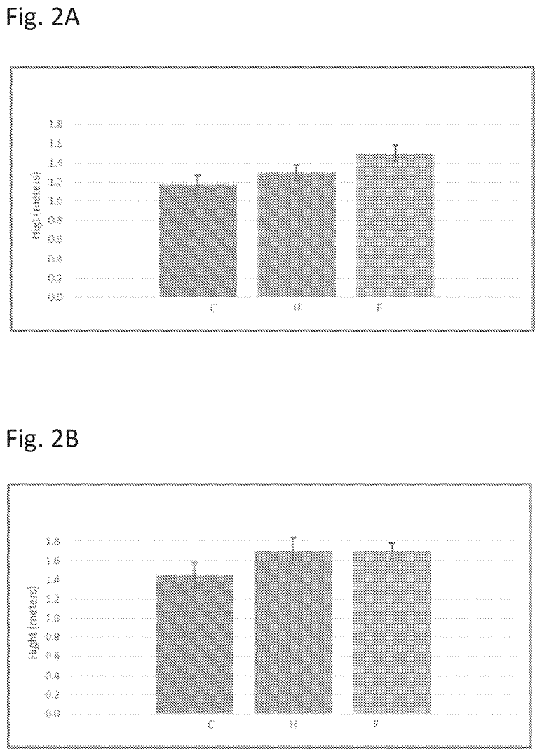

The height of the growing maize plants was measured at two timepoints: Oct. 23, 2017 and Oct. 29, 2017. As may be seen from the above table and from FIGS. 2A (October 23) and 2B (October 29), the plants subjected to treatments H and F were significantly taller than those in the untreated control group.

### 3. Silking

| | | C | H | F |
|---|---|---|---|---|
| 23 Oct. 2017 | Average | 0.00 | 3.50 | 4.00 |
| | SD | 0.00 | 1.29 | 0.82 |
| 29 Oct. 2017 | Average | 20.50 | 24.25 | 48.75 |
| | SD | 1.29 | 1.26 | 2.99 |

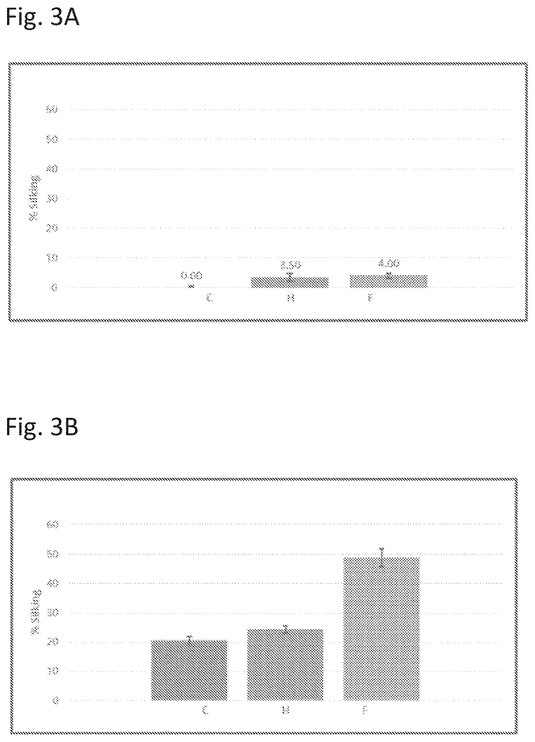

As may be seen from these tabulated results obtained on Oct. 23, 2017 and Oct. 29, 2017, and from FIGS. 3A and 3B, the percentage of the plants which showed signs of silking (i.e. the development of functional stigmas in the female flowers) was significantly higher in the two treatment groups (H and F) than in the untreated control group (C).

### 4. Male Flowering

| | | C | H | F |
|---|---|---|---|---|
| 23 Oct. 2017 | Average | 0.00 | 7.50 | 11.25 |
| | SD | 0.01 | 2.89 | 2.50 |
| 29 Oct. 2017 | Average | 93.00 | 91.00 | 90.75 |
| | SD | 2.16 | 3.37 | 3.77 |

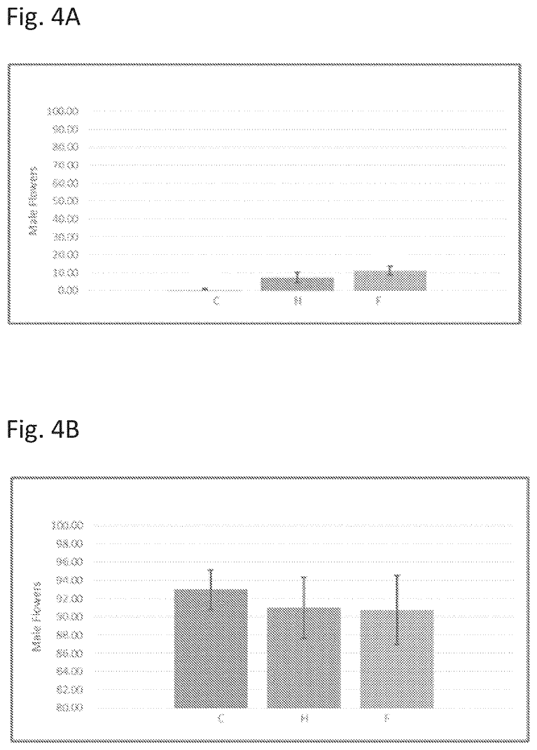

As shown in the upper portion of the above table and the accompanying FIG. 4A, at the first timepoint (October 23), both treatment H and treatment F resulted in a significantly higher degree of male flower formation than in the untreated control treatment (C). At the second timepoint (October 29), however (lower part of the table and FIG. 4B), the degree of male flower formation in the control group was higher than in the treated groups. This indicates a mismatch between the timing of male and female flowering in the control group. In the two treatment groups, however, there is better synchronization of male and female flowering, a situation which is compatible with the development of full kernel yield.

### 5. Second Cob

| | | C | H | F |
|---|---|---|---|---|
| 29 Oct. 2017 | Average | 0.00 | 1.75 | 5.50 |
| | SD | 0.00 | 0.96 | 1.29 |

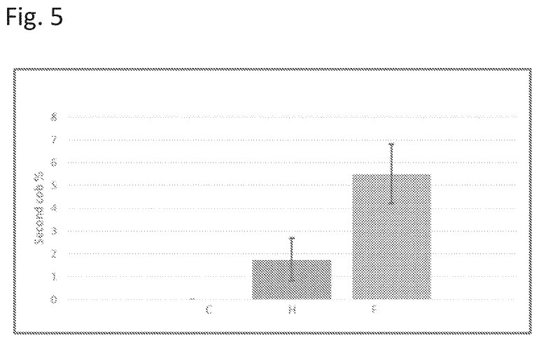

As shown in this table, and summarized graphically in FIG. 5, second cob formation was seen only in the plants in treatment groups H and F, and not in the untreated control plants.

### 6. Foliage Color

| | | C | H | F |
|---|---|---|---|---|
| 23 Oct. 2017 | Average | 1.25 | 7.75 | 8.00 |
| | SD | 0.50 | 0.96 | 0.82 |
| 29 Oct. 2017 | Average | 1.00 | 7.75 | 7.75 |
| | SD | 0.00 | 1.50 | 0.96 |

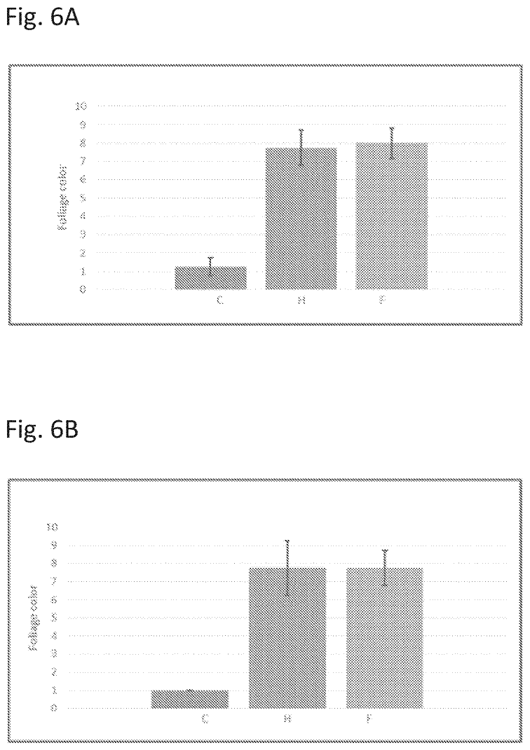

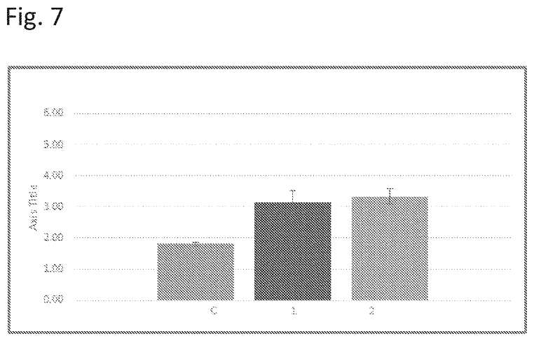

Using a nominal scale of 1-10, the green color of the foliage in the maize plants was assessed on both Oct. 23, 2017 (upper part of table and FIG. 6A) and on Oct. 29, 2017 (lower part of table and FIG. 6B). It will be seen from the results obtained that at both timepoints, there was a significantly greater degree of green color in the plants in treatment groups H and F than in the untreated control. Since the green foliage color is highly associated with the nitrogen availability to the plant, these results provide a further clear indication of the efficacy of treatments H and F in promoting atmospheric nitrogen fixation in maize plants.

### Example 2

### Treatment of Growing Maize Plants with a Composition of the Present Invention: Field Trial 2

### Methods

As in the case of the field study reported in Example 1, above, this trial was sown on the 29 of Aug. 2017 using the pioneer maize silage variety number 32-W-68. The field was washed from possible nitrogen using sprinklers irrigation. The combination treatments administered to the plants which are of relevance for the present study were:
1. Rhizobium leguminosarum biovar viceae, 1% sprayed in the sowing trench and Granules containing the activating agent emulsion together with Calirus and Rhizobium 0.5%.
2. Rhizobium leguminosarum biovar viceae, 1% and the activating agent emulsion sprayed in the sowing slot, and Granules containing the activating agent emulsion together with Calirus and Rhizobium 0.5%.

The amount of the solution containing either the Rhibozium 1% (treatment 1) or Rhizobium 1%, and activating agent (treatment 2) was calculated such that 2 liters per 1000 m row were added to the sowing trench.

In both treatment regimes, the granule quantity was adjusted to 4 Kg granules per 1000m².

The following parameters were monitored at either one timepoint (Oct. 29, 2017) or two timepoints (October 23 and Oct. 29, 2017) during growth of the maize plants:
Nitrogen % in the leaves
Plant height
Flowering (silking and male flowering)
Second cob %
Foliage color

### Results

### 1. Nitrogen Content in the Leaves

| | C | 1 | 2 |
|---|---|---|---|
| Average | 1.80 | 3.16 | 3.33 |
| SD | 0.00 | 0.37 | 0.23 |

As may be seen from the table and from the graph shown in FIG. 7, treatments 1 and 2 result in significantly higher nitrogen levels, compared to the untreated control. These results indicate that these treatments enable the plants to absorb and fix nitrogen from the atmosphere.

### 2. Plant Height

| | | C | 1 | 2 |
|---|---|---|---|---|
| 23 Oct. 2017 | Average | 0.91 | 1.68 | 1.70 |
| | SD | 0.09 | 0.10 | 0.08 |
| 29 Oct. 2017 | Average | 1.46 | 2.18 | 2.00 |
| | SD | 0.05 | 0.10 | 0.08 |

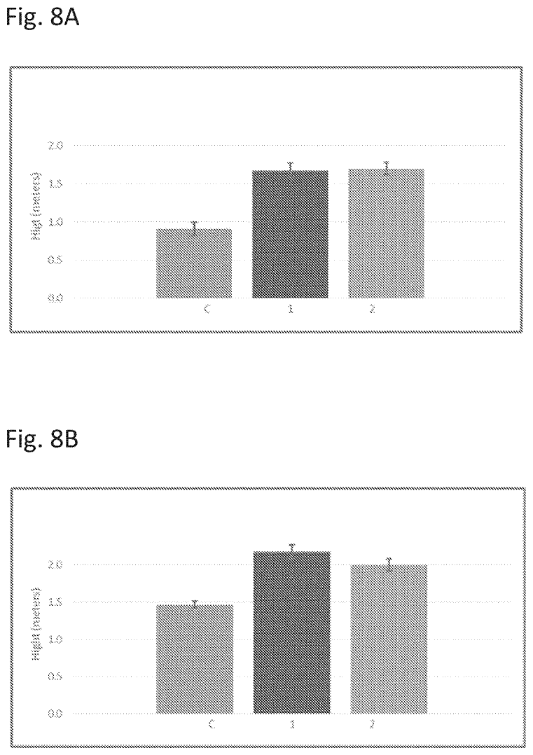

As may be seen from the above table and from FIGS. 8A (October 23) and 8B (October 29), the plants that received treatments 1 and 2 were significantly taller than those in the untreated control group.

### 3. Silking

| | | C | 1 | 2 |
|---|---|---|---|---|
| 23 Oct. 2017 | Average | 0.00 | 5.25 | 11.75 |
| | SD | 0.00 | 0.50 | 2.36 |
| 29 Oct. 2017 | Average | 24.25 | 81.50 | 94.25 |
| | SD | 0.96 | 7.23 | 2.99 |

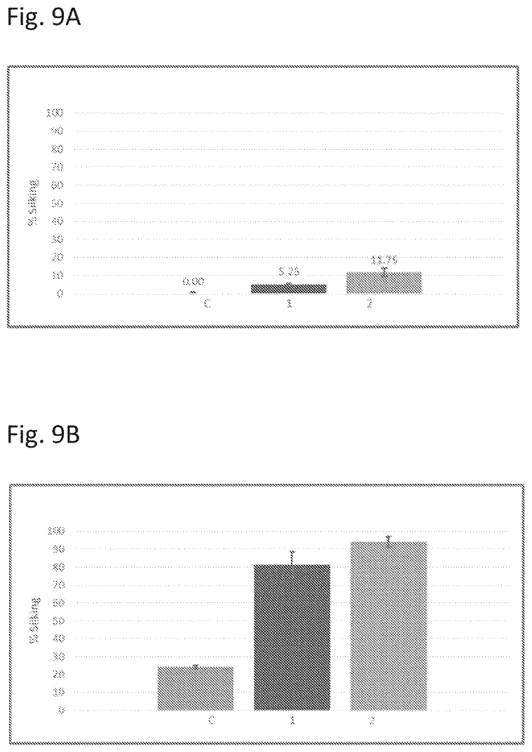

As may be seen from the above table and from FIGS. 9A and 9B, the percentage of the plants which showed signs of silking was significantly higher in both of the two treatment groups (1 and 2) than in the untreated control group (C).

### 4. Male Flowering

| | | C | 1 | 2 |
|---|---|---|---|---|
| 23 Oct. 2017 | Average | 0.00 | 66.25 | 80.00 |
| | SD | 0.00 | 7.50 | 4.08 |
| 29 Oct. 2017 | Average | 96.25 | 100.00 | 100.00 |
| | SD | 4.79 | 0.00 | 0.00 |

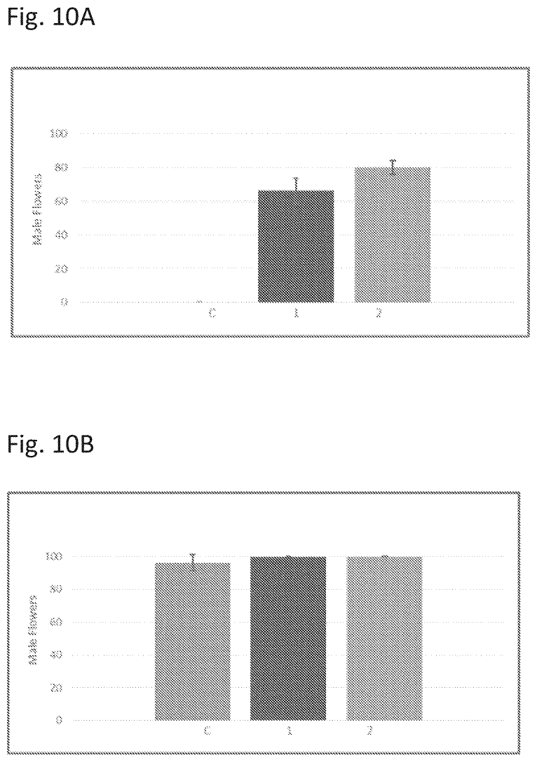

As shown in the upper portion of the above table and the accompanying FIG. 10A, at the first timepoint (October 23), both treatment 1 and treatment 2 resulted in a significantly higher degree of male flower formation than in the untreated control treatment (C). At the second timepoint (October 29), however (lower part of the table and FIG. 10B), the degree of male flower formation in the control group was approximately the same as in the treated groups. In view of the low level of silking (i.e. female flowering) seen at this timepoint (see FIG. 9B), there would appear to be a mismatch between the timing of male and female flowering in the control group. In the two treatment groups, however, there is better synchronization of male and female flowering, a situation which is compatible with the development of full kernel yield.

### 5. Second Cob

| | | C | 1 | 2 |
|---|---|---|---|---|
| 29 Oct. 2017 | Average | 0.00 | 22.75 | 47.50 |
| | SD | 0.00 | 2.22 | 13.23 |

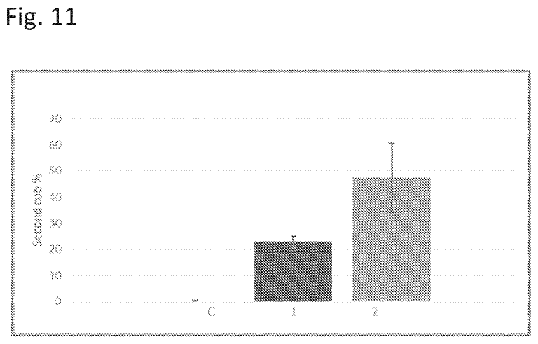

As shown in this table, and summarized graphically in FIG. 11, second cob formation was seen only in the plants in treatment groups 1 and 2, and not in the untreated control plants.

### 6. Foliage Color

| | | C | 1 | 2 |
|---|---|---|---|---|
| 23 Oct. 2017 | Average | 1.25 | 8.00 | 8.75 |
| | SD | 0.50 | 0.82 | 0.50 |
| 29 Oct. 2017 | Average | 1.00 | 7.75 | 8.50 |
| | SD | 0.00 | 0.96 | 0.58 |

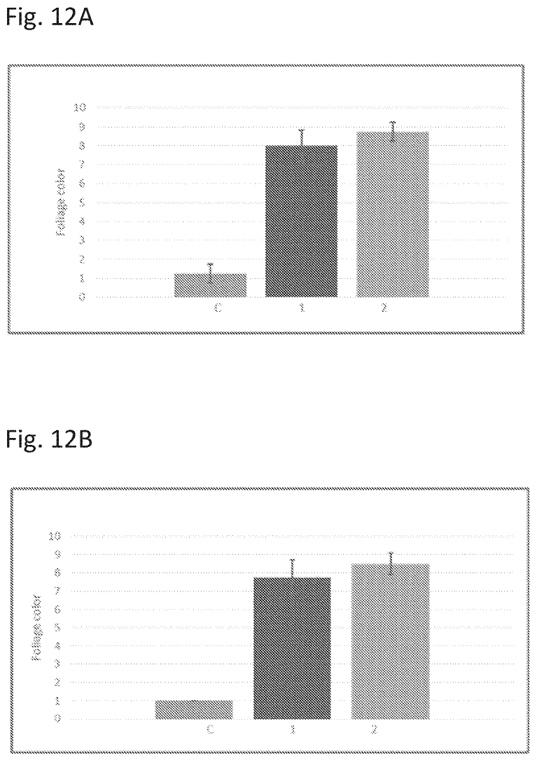

Using a nominal scale of 1-10, the green color of the foliage in the maize plants was assessed on both Oct. 23, 2017 (upper part of table and FIG. 12A) and on Oct. 29, 2017 (lower part of table and FIG. 12B). It will be seen from the results obtained that at both timepoints, there was a significantly greater degree of green color in the plants in treatment groups 1 and 2 than in the untreated control. Since the green foliage color is highly associated with the nitrogen availability to the plant, these results provide a further clear indication of the efficacy of treatments H and F in promoting atmospheric nitrogen fixation in maize plants.

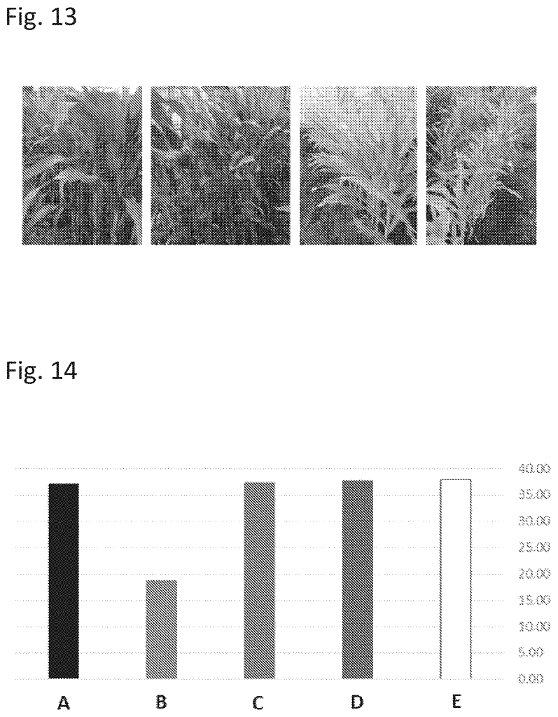

This difference in green coloration and general vitality of the plants between the two treatment groups and the untreated control is also clear in the comparative photograph shown in FIG. 13. Thus, as seen in that figure, the plants subjected to either treatment 1 or treatment 2 have a deeper green color and a much healthier overall appearance than the untreated control plants.

### Example 3

### Treatment of Growing Maize Plants with a Composition of the Present Invention: Field Trial 3—Growth Parameters

### Methods

This trial was sown during the Israeli growing season of 2018 using the pioneer maize silage variety number W86. The field was washed from possible nitrogen using sprinklers irrigation. The combination treatments administered to the plants which are of relevance for the present study were:
A. Positive Control—full commercial nitrogen. The plants were treated with 30 units of nitrogen per 1000 m² by means of applying to this area 60 kg urea containing 46% urea.
B. Negative Control—no added nitrogen.
C. Rhizobium leguminosarum biovar viceae, 3% (containing 10⁹ organisms) was added to the activating agent emulsion described in ‘general methods’ hereinabove, to which Calirus (1%) was also added. Perlite granules were soaked with this mixture as described hereinabove. The granules were added to the sowing trench at a density of 2 Kg granules per 1000m².
D. As for treatment C, but with the granule quantity adjusted to 1 Kg granules per 1000 m².
E. As for treatment C, but with the granule quantity adjusted to 4 Kg granules per 1000 m².

The following parameters were monitored at one timepoint during growth of the maize plants, 3 months after they were sown:
Plant stem caliber
Leaf width
Cob weight (taken from the main stems of 10 plants)
Total plant weight (10 plants)
Plant height

The statistical significance of the difference between the various treatment groups was determined using the Tukey-Kramer HSD test.

### Results

### 1. Plant Caliber

The mean caliber for each of the 5 treatments (A-E) listed above was recorded, and the results obtained are shown below and in FIG. 14:

| Treatment | Mean Plant stem caliber (cm) |
|---|---|
| A. Positive control - full nitrogen | 37.20 |
| B. Negative control - no nitrogen | 18.73 |
| C. Treatment - 2 Kg granules per 1000 m² | 37.49 |
| D. Treatment - 1 Kg granules per 1000 m² | 37.72 |
| E. Treatment - 4 Kg granules per 1000 m² | 37.90 |

These results indicate that each of the three treatments that contained the composition of the present invention (C-E) permitted the growing maize plants to achieve approximately the same plant thickness as that seen with the full nitrogen positive control (A). Each of the treatment regimes produced a mean plant thickness significantly greater than seen with the negative control plants (B).

### 2. Leaf Width

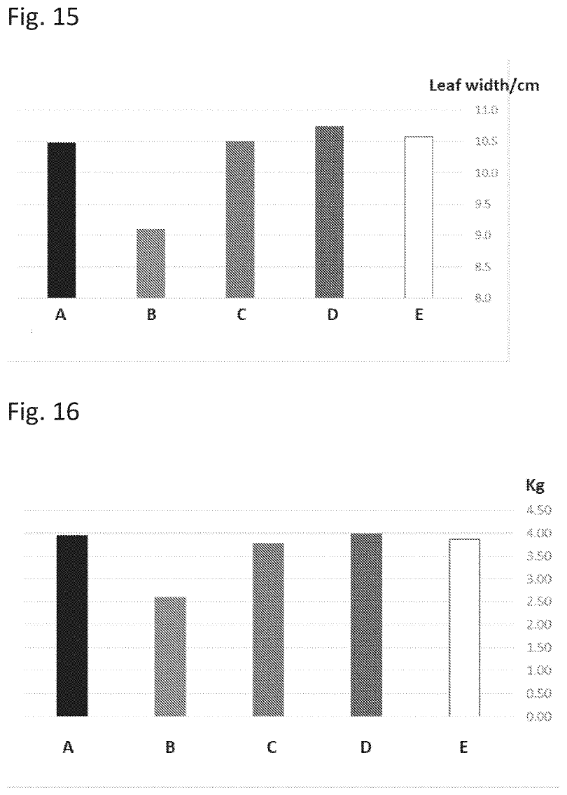

The mean leaf width for each of the 5 treatments (A-E) listed above was recorded, and the results obtained are shown below and in FIG. 15:

| Treatment | Mean leaf width (cm) |
|---|---|
| A. Positive control - full nitrogen | 10.5 |
| B. Negative control - no nitrogen | 9.1 |
| C. Treatment - 2 Kg granules per 1000 m² | 10.5 |
| D. Treatment - 1 Kg granules per 1000 m² | 10.7 |
| E. Treatment - 4 Kg granules per 1000 m² | 10.6 |

These results indicate that each of the three treatments that contained the composition of the present invention (C-E) permitted the growing maize plants to achieve approximately the same mean leaf width as that seen with the full nitrogen positive control (A). Each of the treatment regimes produced a mean leaf width significantly greater than seen with the negative control plants (B).

### 3. Cob Weight (Taken from the Main Stems of 10 Plants)

The mean cob weight from the main stems of 10 plants for each of the 5 treatments (A-E) listed above was recorded, and the results obtained are shown below and in FIG. 16:

| | Mean cob weight |
|---|---|
| Treatment | (10 plants; kg) |
| A. Positive control - full nitrogen | 3.96 |
| B. Negative control - no nitrogen | 2.61 |
| C. Treatment - 2 Kg granules per 1000 m² | 3.78 |
| D. Treatment - 1 Kg granules per 1000 m² | 3.98 |
| E. Treatment - 4 Kg granules per 1000 m² | 3.86 |

These results indicate that each of the three treatments that contained the composition of the present invention (C-E) resulted in approximatley the same mean cob weight as that seen with the full nitrogen positive control (A). Each of the treatment regimes produced a mean cob weight significantly greater than seen with the negative control plants (B).

### 4. Total Plant Weight (10 Plants)

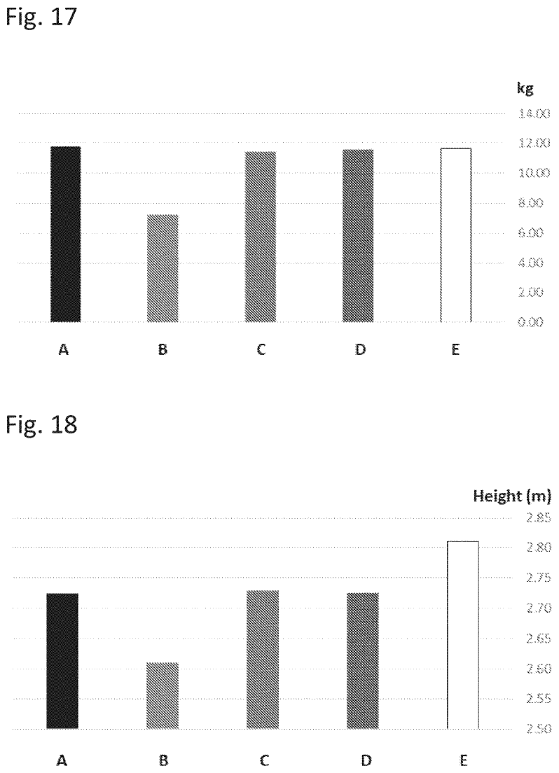

The mean total plant weight of 10 plants for each of the 5 treatments (A-E) listed above was recorded, and the results obtained are shown below and in FIG. 17:

| | Total plant weight |
|---|---|
| Treatment | (10 plants; kg) |
| A. Positive control - full nitrogen | 11.81 |
| B. Negative control - no nitrogen | 7.21 |
| C. Treatment - 2 Kg granules per 1000 m² | 11.46 |
| D. Treatment - 1 Kg granules per 1000 m² | 11.62 |
| E. Treatment - 4 Kg granules per 1000 m² | 11.65 |

These results indicate that each of the three treatments that contained the composition of the present invention (C-E) resulted in approximately the same mean total plant weight as that seen with the full nitrogen positive control (A). Each of the treatment regimes produced a mean total plant weight that was significantly greater than seen with the negative control plants (B).

### 5. Plant Height

The mean plant height of 10 plants seen with each of the 5 treatments (A-E) listed above was recorded, and the results obtained are shown below and in FIG. 18:

| Treatment | Plant height (m) |
|---|---|
| A. Positive control - full nitrogen | 2.72 |
| B. Negative control - no nitrogen | 2.61 |
| C. Treatment - 2 Kg granules per 1000 m² | 2.73 |
| D. Treatment - 1 Kg granules per 1000 m² | 2.73 |
| E. Treatment - 4 Kg granules per 1000 m² | 2.81 |

These results indicate that each of the three treatments that contained the composition of the present invention (C-E) resulted in approximately the same mean total plant weight as that seen with the full nitrogen positive control (A). Although each of the treatment regimes produced a mean plant height that was slightly greater than seen with the negative control plants (B), this difference did not reach statistical significance.

### Example 4

### Treatment of Growing Maize Plants with a Composition of the Present Invention: Field Trial 4—Total Leaf Nitrogen Content

### Methods

This trial was sown during the Israeli growing season of 2018 using the pioneer maize silage variety number W86. The field was washed from possible nitrogen using sprinkler irrigation. The combination treatments administered to the plants which are of relevance for the present study were:
PC. Positive Control—full commercial nitrogen. The plants were treated with 30 units of nitrogen per 1000 m² by means of applying to this area 60 kg urea containing 46% urea.
NC. Negative Control—no nitrogen.
1. R1% G1kg=granules prepared as in ‘general methods’ and in Example 3, above, prepared using a 1% isolate of Rhizobium leguminosarum biovar viceae, applied to the sowing trench at a density of 1 Kg granules per 1000 m².
2. R1% G2kg=as 1 but with 2 kg granules per 1000 m^{2.}
3. R1% G4kg=as 1 but with 4 kg granules per 1000m^{2.}
4. R3% G1kg=as 1 but the granules were prepared with a Rhizobium concentration of 3%.
5. R3% G2kg=as 4 but with 2 kg granules per 1000 m^{2.}
6. R3% G4kg=as 4 but with 4 kg granules per 1000 m²
7. R5% G1kg=as 1 but the granules were prepared with a Rhizobium concentration of 5%.
8. R5% G2kg=as 7 but with 2 kg granules per 1000 m².
9. R5% G4kg=as 7 but with 4 kg granules per 1000m².
10. (R3% G2kg)2=as 5 but the granules were prepared with a Rhizobium concentration of 6%.

The average total leaf nitrogen content was measured at one timepoint during growth of the maize plants, 3 months after sowing.

### Results

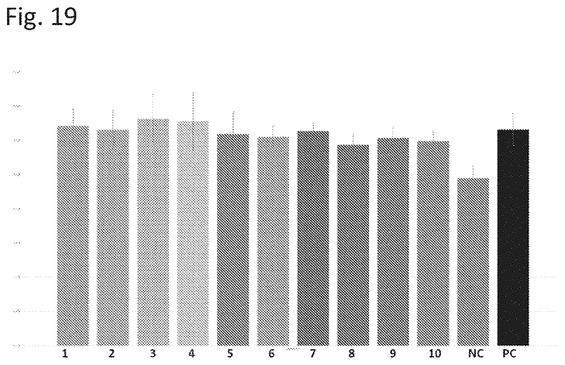

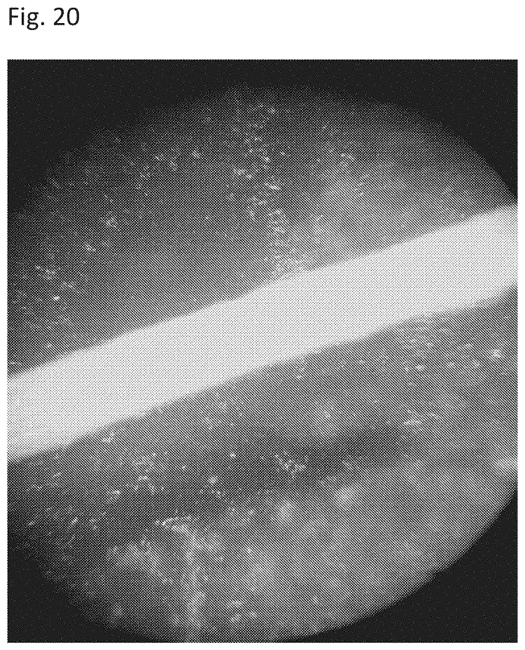

The average total nitrogen content of the leaves was measured, and the results shown in the following table and in FIG. 19.

| TREATMENT | AVERAGE N2 CONTENT | S.D. |
|---|---|---|
| 1. R1% G1 kg | 3.22 | 0.25 |
| 2. R1% G2 kg | 3.16 | 0.28 |
| 3. R1% G4 kg | 3.31 | 0.37 |
| 4. R3% G1 kg | 3.28 | 0.42 |
| 5. R3% G2 kg | 3.09 | 0.33 |
| 6. R3% G4 kg | 3.05 | 0.16 |
| 7. R5% G1 kg | 3.14 | 0.11 |
| 8. R5% G2 kg | 2.94 | 0.16 |
| 9. R5% G4 kg | 3.04 | 0.16 |
| 10. (R3% G2 kg)2 | 2.99 | 0.16 |
| NC. Negative Control - | 2.45 | 0.18 |
| no nitrogen. | | |
| PC. Positive Control - | 3.16 | 0.24 |
| full commercial nitrogen. | | |

These results indicate that each of the various treatments with the composition of the present invention resulted in nitrogen levels within the maize plants that were comparable with those obtained with the positive control (PC). Each of these treatments resulted in significantly higher leaf nitrogen levels than those seen in the untreated control group (NC). It may thus be concluded that treatment with the composition of the present invention allows Rhizobium bacteria to cause nitrogen fixation in growing maize plants.

### Example 5

### Treatment of Growing Wheat with a Composition of the Present Invention: Field Trial 5

Two different agricultural sites in Israel were selected for field trials in which the effects of compositions of the present invention on wheat crops were investigated. The various compositions were administered to the growing wheat (Galil variety) as described in Examples 3 and 4, hereinabove. The treatments used in this study are as follows:

B. Negative control (no nitrogen source)

A. Granules prepared according to Example 3, applied at a density of 4 kg granules per 1000 m².

C. Granules prepared according to Example 3, applied at a density of 2 kg granules per 1000 m².

F. Positive control—full commercial nitrogen. The plants were treated with 30 units of nitrogen per 1000 m² by means of applying to this area 60kg urea containing 46% urea.

### Results

### 1. Appearance of Roots Following Treatment

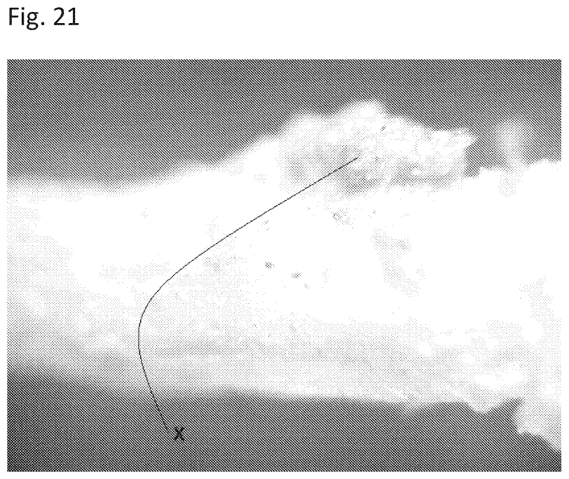

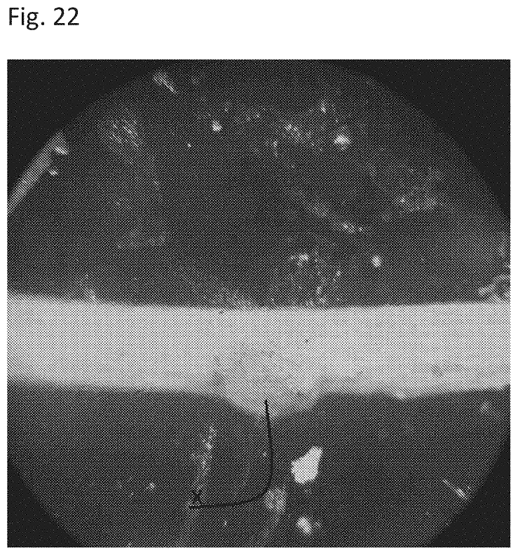

In the absence of treatment with granules containing a composition of the present invention, the roots of the growing wheat plants did not show any evidence of root nodule formation. This is seen in FIG. 20, which shows (in white) a smooth elongate root with no signs of nodule or glomerule formation. By way of comparison, FIG. 21 presents a photograph of the root of a plant that has been subjected to treatment A (i.e. granules containing the composition of the present invention at a dosage of 4 kg granules per 1000 m².) It may be seen from this figure that a rough nodule (X) has formed on one side of the root. Similarly, FIG. 22 shows the formation of a root nodule (X) on the site of the root of a wheat plant subjected to treatment with treatment C (granules containing the composition of the present invention at a dosage of 2 kg granules per 1000 m².)

Nodule development in these samples indicates the possible site of a symbiotic relationship between the administered Rhizobium bacteria and the plant root system, which has developed as part of the nitrogen fixation process induced by the treatment with the composition of the present invention.

### 2. Effect of the Various Treatments on Wheat Plant Parameters

The following parameters were measured in the wheat, in order to assess the effect of the treatment compositions on plant growth:

a) Number of side shoots;

b) Flag leaf width;

C) Main shoot diameter.

The results of these measurements are presented in the table, below:

| | | A | C | F |
|---|---|---|---|---|
| | | (4 kg | (2 kg | (positive |
| | B | granules | granules | control; |
| | (negative | per 1000 | per 1000 | added |
| Treatment: | control) | m²) | m²) | Nitrogen) |
| No. of side | 1.18 | 1.6 | 2.09 | 1.3 |
| shoots | | | | |
| Flag leaf width | 1.7 | 2.1 | 2.2 | 2.0 |
| (cm) | | | | |
| Main shoot | 0.33 | 0.48 | 0.5 | 0.42 |
| diameter (cm) | | | | |

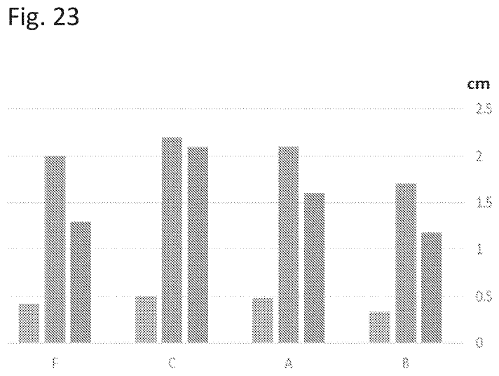

These data are also presented graphically in FIG. 23. In that figure, the results for main shoot diameter are given in the left bar of each treatment, the flag leaf width data is given in the middle bar and the number of side shoots in the right bar.

It may be seen from these results that all of the measured growth parameters are increased following treatment with either treatment A or treatment C, in relation to the negative control. In addition, said treatments also provide growth results either comparable with, or greater than, those obtained with the positive control.

### 3. Effect of the Various Treatments on Nitrogen Fixation in Flag Leaves of Wheat Plants

The results for flag leaf nitrogen fixation are presented in the following table:

| Treatment | Average nitrogen content | SD |
|---|---|---|
| A. 4 kg granules | 2.94 | 0.07 |
| B. Negative control | 2.44 | 0.09 |
| C. 2 kg granules | 2.96 | 0.19 |
| F. Positive control | 2.94 | 0.20 |

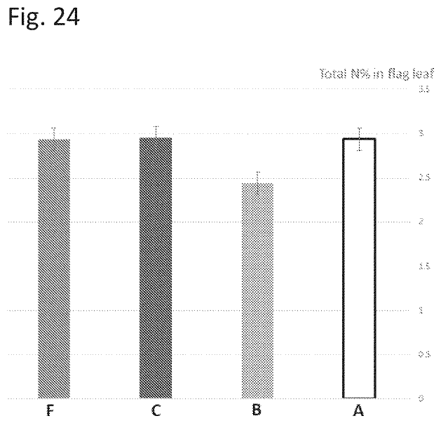

These results are also summarized graphically in FIG. 24.

It may be seen from these results that both treatments A and C (compositions of the present invention) and the positive control caused an increase in flag leaf nitrogen content, as compared with the negative control. Both of these treatment regimens resulted in increases in nitrogen content similar to those caused by the positive control.

### 4. Effect of the Various Treatments on Grain Yield

The following table summarizes the results for the effect of the treatments and controls on average wheat grain yield from each of 6 2 m plots:

| Treatment | Average wheat grain yield (kg) |
|---|---|
| A (4 kg granules) | 1.27 |
| B (negative control) | 1.06 |
| C (2 kg granules) | 1.08 |
| F (positive control - | 1.11 |
| added nitrogen) | |

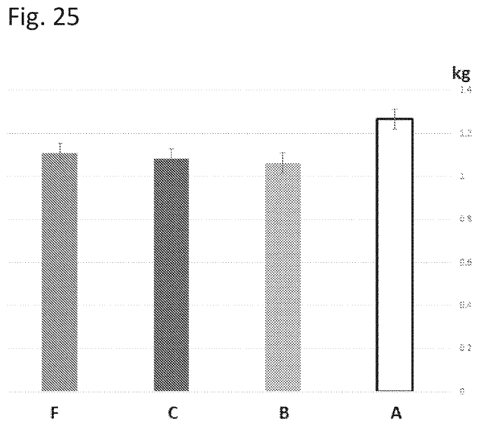

These data are also presented in the form of a graph in FIG. 25.

It may be seen from these results that both of the treatments containing a composition of the present invention and the positive control caused a significant increase in wheat grain yield in this field study (i.e. compared with negative control). The increase due to the two treatment regimens was numerically similar to that caused seen in the positive control group.

### Field Trials—Conclusions

In all of the field trials reported hereinabove (Examples 1-5), the treatment regimens comprising combinations of both Rhizobium and an emulsion of the mixture of activating agents resulted in increased fixation of atmospheric nitrogen, as witnessed by the direct measurement of foliar nitrogen levels, the development of root nodules and the various growth-related parameters measured in these trials. This positive effect was seen regardless of the way in which the treatment combinations were administered.

### Example 6

### Initial Screening of Phytochemicals for Their Potential Use as Activating Agents for Rhizobium Species

### Introduction

Cucumber (Cucumis sativus L) seedlings are highly susceptible to fungal and bacterial pathogens attacking the seedling during the germination process and were therefore selected as a model plant to screen and calibrate the Rhizobium species and the phytochemicals that can cause activation thereof.

### Material and Methods

### 1. Phytochemical Screening

The potential phytochemicals were added to a mixture of 30 cc glucose 50% V/V substrate, 10 cc cocktail of fungal pathogens and 10 cc cocktail of bacterial pathogens in a Petri dish. The fungal cocktail contained: Botrytis cinerea, Rhizoctonia solani, Pythium spp. and non-pathogenic fungi used for the fermentation of tomatoes. The bacterial cocktail contained: Clavibacter michiganensis, Xanthomonas campestris, Pseudomonas syringae and non-pathogenic bacteria used for the fermentation of tomatoes.

Approximately 1000 potential phytochemicals were screened for their ability to activate, Rhizobium species by means of calculating a colony forming index for each test (0=no colony; 5=maximal colony size). The five phytochemicals listed above in the introduction to the Examples section were selected from the approximately 1000 phytochemicals tested on the basis of their superior performance as activating agents for Rhizobium species.

The optimal combination and concentrations of the five selected activating agents listed above were determined for each of the host organisms used in the studies reported below. The selected combinations were those found in preliminary studies to have the lowest possible concentrate that was capable of producing the desired protective effect. In this way, possible side effects and environmental pollution during the administration of these agents to the host organisms were avoided.

At the same time the phytochemicals were screened for their ability to eliminate a cocktail of bacterial and fungal pathogens. For the purposes of comparison between the various treatments, fungal and bacterial elimination indices were calculated (0=maximal elimination, 5=no elimination).

The test mixtures, containing the glucose substrate and fungal and bacterial cocktails mentioned above together with all five of the activating phytochemicals and a penetrator (MCT) and two type of surface active agent sugar ester and iso lecithin were used at four different concentrations: concentrations 1, 2, 3 and 4. In each case, the same amount of glucose substrate and fungal and bacterial cocktails—30 ml—was added to the mixture. Similarly, the concentration of the MCT and surface active agent were correlated to the concentration of the actives if Sclareol content at concentration 2 doubled then MCT and surface active agent concentrations were also doubled, and so on. However, the concentrations of the Rhizobium species and each of the five activating agents (given in %) were 3%. at concentration 1, 3 and 5% at concentration 2,4 as described in the Table I:

TABLE I

| | Concen- | Concen- | Concen- | Concen- |
|---|---|---|---|---|
| | tration 1 | tration 2 | tration 3 | tration 4 |
| Rhizobium | 3% | 5% | 3% | 5% |
| species | | | | |
| Sclareol | 0.04% | 0.08% | 0.12% | 0.2% |
| 98% | | | | |
| Naringin | 0.04% | 0.08% | 0.12% | 0.2% |
| 98% | | | | |
| Nootkatone | 0.04% | 0.08% | 0.12% | 0.2% |
| 98% | | | | |
| Stevia | 0.005% | 0.01% | 0.014% | 0.035% |
| CBD* 100% | 0.001% | 0.002% | 0.003% | 0.005% |

*hemp oil containing 15% CBD

Various different test mixtures containing different combinations of some or all of the five activating agents were used in this study, in accordance with the list of treatments given in Table II, below. In each case, the activating agents, Rhizobium species and substrate were used at the concentrations indicated in Table I. For example, when tested at Concentration 1, the concentration of sclareol in test mixtures containing that activating agent was 0.04%, while when tested at Concentration 2, sclareol was present at a concentration of 0.08%, and so on.

TABLE II

| | | Rhizobium | | | | | Hemp |
|---|---|---|---|---|---|---|---|
| TEST | Substrate | species | Sclareol | Naringin | Nootkatone | Stevia | Oil |
| 1 | ✓ | — | — | — | — | — | — |
| 2 | ✓ | ✓ | — | — | — | — | — |
| 3 | ✓ | ✓ | ✓ | — | — | — | — |
| 4 | ✓ | ✓ | — | ✓ | — | — | — |
| 5 | ✓ | ✓ | — | — | ✓ | — | — |
| 6 | ✓ | ✓ | — | — | — | ✓ | — |
| 7 | ✓ | ✓ | — | — | — | — | ✓ |
| 8 | ✓ | ✓ | ✓ | ✓ | — | — | — |
| 9 | ✓ | ✓ | ✓ | ✓ | ✓ | — | — |
| 10 | ✓ | ✓ | ✓ | ✓ | ✓ | ✓ | — |
| 11 | ✓ | ✓ | ✓ | ✓ | ✓ | ✓ | ✓ |
| 12 | ✓ | | ✓ | ✓ | ✓ | ✓ | ✓ |
| 13 | ✓ | | | ✓ | ✓ | ✓ | ✓ |
| 14 | ✓ | | | | ✓ | ✓ | ✓ |
| 15 | ✓ | | | | | ✓ | ✓ |
| 16 | ✓ | | | | | | ✓ |

### Results

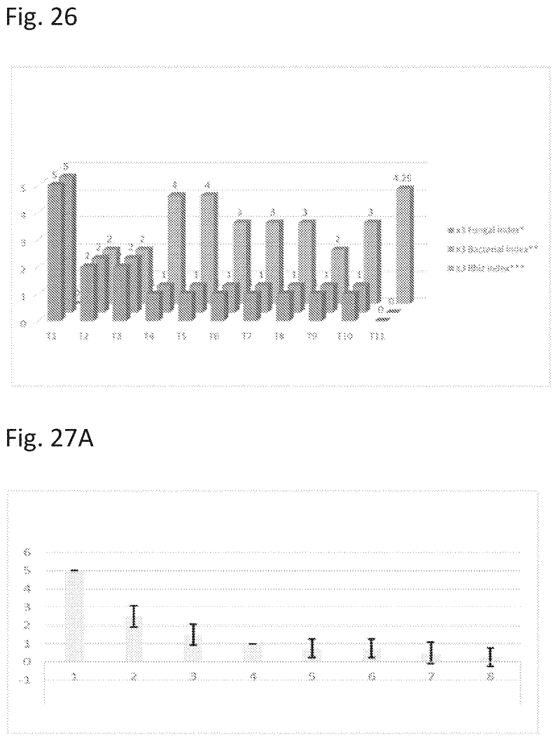

Preliminary results indicated that the optimal anti-fungal and anti-bacterial activity was obtained using test mixtures with concentration 2 and concentration 3 (see table above). Since Rhizobium species colony development was optimal using concentration 3, this was the concentration that was selected for use in the remainder of the study. The results obtained for fungal elimination, bacterial elimination and Rhizobium species activation (colony size) for the concentration 3 tests are summarized graphically in FIG. 26 in the front, middle and back rows of the graph, respectively. The eleven different treatments summarized in Table II, above, are labeled as T1 to T11 along the X axis of the graph.

As explained above, the three semi-quantitative indices used to assess the anti-fungal, anti-bacterial and activation properties are as follows:

Fungal index: 0 (no development) to 5 (maximum development)

Bacterial index: 0 (no development) to 5 (maximum development)

Rhizobium index (colony forming index): 0 (no development) to 5 (maximum development)

It may be seen from FIG. 26 that the best results—both for Rhizobium species activation and for pathogen elimination were obtained using treatment 11, which (as shown in Table II, above) used a combination of all five activation agents.

The identification numbers of the actives:

| | |
|---|---|
| 1 | Pathogen mix |
| 2 | Rhizobium complex |
| 3 | Sclareol 98% |
| 4 | Naringin 98% |
| 5 | Nootkatone 98% |
| 6 | stevia |
| 7 | Hemp Oil contain CBD 15% calculated as 100% CBD |
| 1 | Pathogen mix |
| 2 | 1 + 2 |
| 3 | 1 + 2 + 3 |
| 4 | 1 + 2 + 4 |
| 5 | 1 + 2 + 5 |
| 6 | 1 + 2 + 6 |
| 7 | 1 + 2 + 7 |
| 8 | 1 + 2 + 3 + 4 |
| 9 | 1 + 2 + 3 + 4 + 5 |
| 10 | 1 + 2 + 3 + 4 + 5 + 6 |
| 11 | 1 + 2 + 3 + 4 + 5 + 6 + 7 |
| 12 | 1 + 3 + 4 + 5 + 6 + 7 |
| 13 | 1 + 3 + 4 + 5 + 6 |
| 14 | 1 + 3 + 4 + 5 |
| 15 | 1 + 3 + 4 |
| 16 | 1 + 3 |

### Example 7

### Effect of Altering the Activating Agent Composition on the Activation of Rhizobium Species and the Fungicidal and Bactericidal Activities of Said Composition

A second group of studies was aimed at investigating the effect of either eliminating one phytochemical from the full 5-component combination or of selectively altering the concentration of one or two components in the mixture.

### Material and Methods

As for Example 1.

The various test mixtures were used at either concentration 3 or concentration 4 (as defined in Example 1, above). The composition of each of these test mixtures is summarized in the following two tables:

TABLE III
Concentration 3

| | | Rhizobium | | | | | |
|---|---|---|---|---|---|---|---|
| TEST | Substrate | species | Sclaerol | Naringin | Nootkatone | Stevia | CBD |
| 1 | ✓ | — | — | — | — | — | — |
| 2 | ✓ | ✓ | — | — | — | — | — |
| 3 | ✓ | ✓ | — | ✓ | ✓ | — | ✓ |
| 4 | ✓ | ✓ | ✓ | ✓ | ✓ | — | ✓ |
| 5 | ✓ | ✓ | ✓ | ✓ | — | ✓ | ✓ |
| 6 | ✓ | ✓ | ✓ | — | ✓ | ✓ | ✓ |
| 7 | ✓ | ✓ | — | ✓ | ✓ | ✓ | ✓ |
| 8 | ✓ | ✓ | ✓ | ✓ | ✓* | ✓* | ✓ |

*In test 8, the Nootkatone and Stevia were each present at an elevated concentration - 0.4% v/v Nootkatone (instead of 0.3%) and 1.0% Stevia (instead of 0.75%).

TABLE IV
Concentration 4

| | | Rhizobium | | | | | |
|---|---|---|---|---|---|---|---|
| TEST | Substrate | species | Sclaerol | Naringin | Nootkatone | Stevia | CBD |
| 1 | ✓ | — | — | — | — | — | — |
| 2 | ✓ | ✓ | — | — | — | — | — |
| 3 | ✓ | ✓ | — | ✓ | ✓ | — | ✓ |
| 4 | ✓ | ✓ | ✓ | ✓ | ✓ | — | ✓ |
| 5 | ✓ | ✓ | ✓ | ✓ | — | ✓ | ✓ |
| 6 | ✓ | ✓ | ✓ | — | ✓ | ✓ | ✓ |
| 7 | ✓ | ✓ | | ✓** | ✓ | ✓ | ✓ |

**In test 7, the Naringin was present at a reduced concentration - 0.3% v/v (instead of 0.4%).

### Results

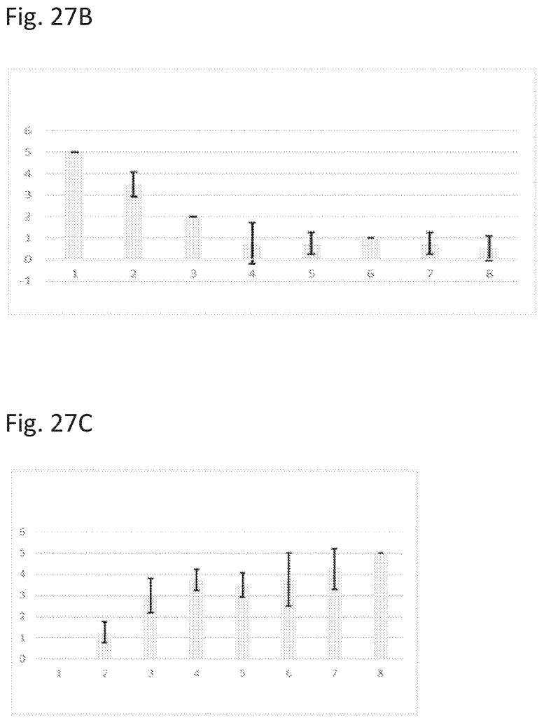

As may be seen in FIGS. 27A, 27B and 27C, all test mixtures containing 3 or 4 activating agents used at concentration 3 caused significant reduction in the fungal and bacterial indices and a significant increase in the Rhizobium species activation index, when compared with medium only and medium plus Rhizobium species controls (mixtures 1 and 2, respectively).

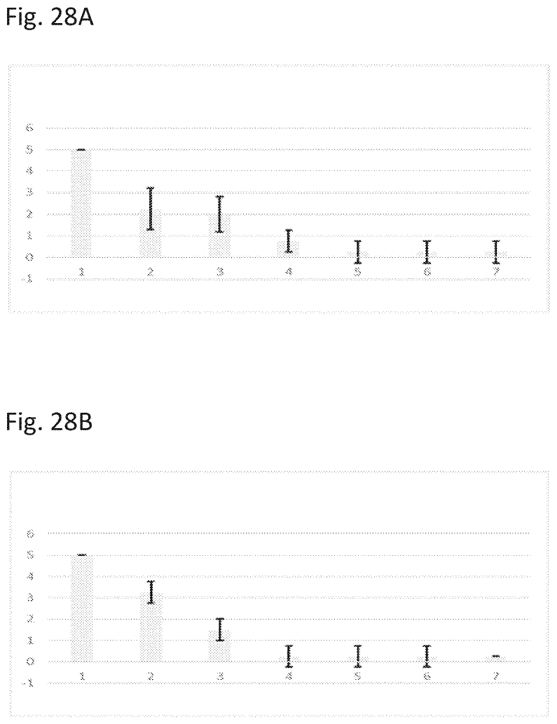

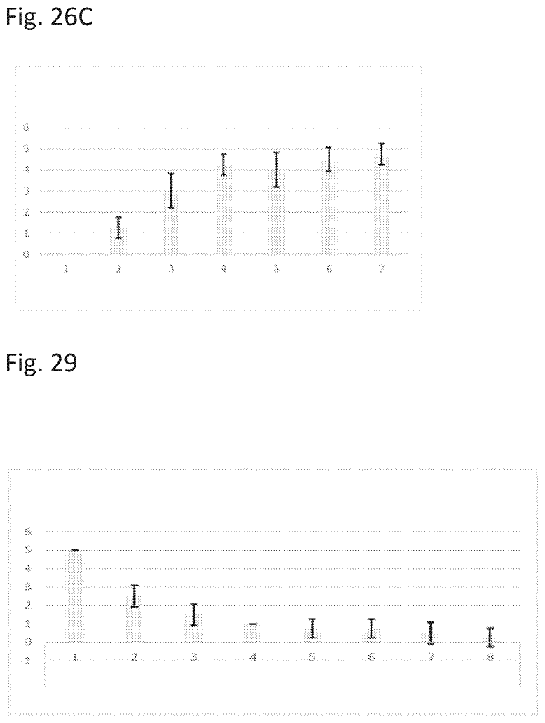

Similarly, as shown in FIGS. 28A, 28B and 28C, all test mixtures containing 3 or 4 activating agents used at concentration 4 caused significant reduction in the fungal and bacterial indices and a significant increase in the Rhizobium species activation index, when compared with medium only and medium plus Rhizobium species controls (mixtures 1 and 2, respectively).

It may also be observed in FIGS. 28A, 28B and 28C that the five-component activating agent mixture in which the Nootkatone and Stevia components are both at an elevated concentration (i.e. concentration 4, while all other components are at concentration 3; i.e. test mixture 8) has the greatest activity on all three indices.

Furthermore, FIGS. 28A, 28B and 28C show that the four-component activating agent mixture (number 7) in which the naringin concentration is reduced to concentration 3, with all other components at concentration 4, has the greatest activity in this data set, as measured by all three indices.

These data indicate that mixtures containing less than the maximum five activating agents may be used to protect host organisms from fungal or bacterial attack. In addition, these results also indicate that optimization of the mixtures may be obtained by manipulating the concentration of one or more individual activating agents in the mixture.

### Example 8

### The Fungicidal and Bactericidal Activities of Various Activating Agent Compositions in Conjunction With a Different Rhizobium Species Formulation

In this study, the experiments performed in Example 7, above, were repeated using a different Rhizobium species preparation, namely a Rhizobium composition produced and sold by Bio-Lab Ltd., Jerusalem, Israel labeled as “Culture for growing groundnuts”.

### Material and Methods

As for Example 6.

The various test mixtures were used at either concentration 3 or concentration 4 (as defined in Example 6, above). The composition of each of these test mixtures is as summarized in Tables III and IV in Example 7, hereinabove.

### Results

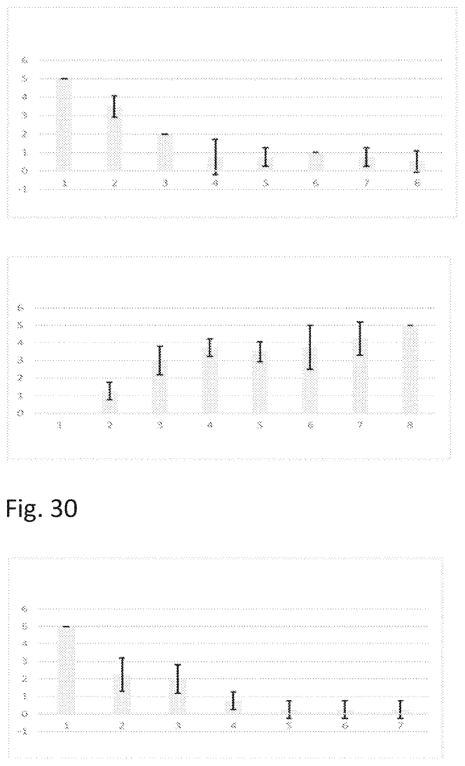

This study confirms the results obtained in Example 7. Thus, as seen in FIG. 29 (concentration 3) and FIG. 30 (concentration 4), all test mixtures containing 3, 4 or 5 activating agents at concentration 3, caused a marked reduction in the fungal and bacterial indices. Furthermore, they also caused a significant increase in the Rhizobium species activation index.

Of particular note is the fact that at concentration 3, the five-component activating agent mixture in which the Nootkatone and Stevia components are both at an elevated concentration (i.e. concentration 4, while all other components are at concentration 3; i.e. test mixture 8) has the greatest activity on all three indices (FIG. 29). Similarly, as shown in FIG. 30, the four-component activating agent mixture (number 7) in which the naringin concentration is reduced to concentration 3, with all other components at concentration 4, has the greatest activity in this data set, as measured by all three indices.

These results, obtained with the Rhizobium species formulation confirm the findings obtained with the formulation (Example 7, hereinabove), indicating that the effects observed are not specific to any one particular Rhizobium preparation.

### Example 9

### Anti-Inflammatory Activity of Agents Used in the Present Invention

Following the results obtained with combinations of Rhizobium species and some or all of the five activating agents reported in Examples 6-8, hereinabove, said agents were investigated in order to look for common functional properties, in addition to their bactericidal, fungicidal and Rhizobium species—activating abilities.

Following a series of preliminary investigations, the present inventors unexpectedly found that each of the five activating agents tested in the studies presented hereinabove, also share a highly potent anti-inflammatory activity.

In order to investigate this further, three of the activating agents used in the previous Examples—both separately, in combination with each other and in combination with Rhizobium species—, were tested for their ability to inhibit the in vitro production in a cultured macrophage line of two key inflammatory inhibitors: nitric oxide (NO) and TNF-α. In addition, the viability of the macrophages was measured at appropriate IC₅₀ values corresponding to the inhibition of NO and TNF-α, at the time that the anti-inflammatory assays were performed.

### Methods

### RAW 264.7 Macrophage Cell Line

RAW 264.7 macrophages were grown in flat-bottomed flasks using a standard growth medium (DMEM supplemented with 5% FBS, antibiotics and glutamine. The cells were maintained in accordance with standard procedures well known in the art. After the cells reached confluence, they were removed from the flasks using mechanical means and then concentrated by centrifuging and resuspended in a small volume of fresh culture medium. The cell concentration was adjusted with growth medium in order that about 75,000 cells could be added to each well of a 96-well plate. A combination of 25 μg/mL LPS and 10 U/ml IFN-γ DMEM, was used for activation of the macrophages. The various test agents were added to the wells one hour prior to activation. The cells were then incubated for a further 24 hours, prior to assaying the inflammatory mediator production and cell viability.

### Determination of Cell Viability

The Alamar Blue assay of viability was performed by adding 100 μl of a 10% Alamar Blue solution to each well and incubating at 37° C. for 1-2 hr. Fluorescence was measured (excitation at 545 nm and emission at 595 nm) and expressed as a percentage of the values in untreated control cells.

### Determination of Nitric Oxide Production by Griess Assay

The production of NO by the macrophages subjected to the various treatments was assayed using the Griess reagent (equal volumes of 1% sulphanilamide and 0.1% napthyethylene-diamine in 5% HCl). 70 μl of supernatant from each test well was transferred to a fresh 96-well plate and mixed with 70 μL of Griess reagent and the violet color produced was measured at 540 nm.

### TNF-α Determination by ELISA

A sandwich ELISA was used to determine TNF-α concentration. The primary antibody was used at a concentration of 0.5 μg/mL in PBS. Serial dilutions of TNF-α standard from 0 to 1000 pg/mL in diluent (0.05% Tween-20, 0.1% BSA in PBS) were used as internal standard. TNF-α was detected with a biotinylated second antibody and an avidin peroxidase conjugate with TMB as detection reagent. The color development was monitored at 655 nm, taking readings after every 5 minutes. After 25 minutes, the reaction was stopped using 0.5 M sulphuric acid and the absorbance was measured at 450 nm.

### Tested Agents

The methods described above were used to determine the effects of Sclareol, Naringin and Steviol, and their combinations with each other and with Rhizobium species—, on NO and TNF-α production, and on cell viability. The results for the anti-inflammatory activities are presented as IC₅₀ values for the inhibition o NO and TNF-α production in Table V, below, together with the cell viability results. In addition, comparable results obtained from the scientific literature (A. S. Ravipati et al. (2012) BMC Complementary and Alternative Medicine, 12:173 “Antioxidant and anti-inflammatory activities of selected Chinese medicinal plants and their relation with antioxidant content”) for two additional plant species—aqueous extracts of Aster tataricus and Cyperus rotundus—are presented at the end of the table. Extracts of these two species were investigated by the present inventors with regard to their fungicidal and bactericidal effects in combination with B. subtilis. The results of these investigations are presented in Example 5, hereinbelow.

### Results

The results obtained for the anti-inflammatory and viability assays of the cultured macrophages treated with the various agents are presented in Table V, below.

TABLEV

| | IC₅₀ for the | | IC₅₀ for the | |
|---|---|---|---|---|
| Name of agent/ | Inhibition of | Cell viability | inhibition of | Cell viability |
| combination/plant | NO production | (% of cell | TNF-α production | (% of cell |
| extract tested | (mg/ml) | survival) | (mg/ml) | survival) |
| 1. Sclareol | 0.04 ± 0.02 | 96.20 ± 2.1 | 0.08 ± 0.02 | 95.30 ± 2.2 |
| 2. Naringin | 0.04 0.02 | 90.10 ± 4.2 | 0.09 ± 0.02 | 91.50 ± 5.2 |
| 3. Steviol | 0.06 ± 0.03 | 98.20 ± 4.2 | 0.08 ± 0.02 | 99.54 ± 4.1 |
| 1 + 2 + 3 | 0.004 ± 0.002 | 95.76 ± 4.5 | 0.01 ± 0.003 | 92.36 ± 2.1 |
| Rhizobium species | NA | 89.5 ± 3.5 | NA | 86.55 ± 2.0 |
| Aster tataricus | 0.14 ± 0.08 | 98.95 ± 1.5 | 2.30 ± 0.09 | 99.70 ± 0.5 |
| Cyperus rotundus | 0.35 ± 0.37 | 86.60 ± 19 | 2.39 ± 0.64 | 107.50 ± 10.6 |

It may be seen that none of the treatment agents tested had any significant adverse effect on the viability of the macrophages. Consequently, any inhibition of the production of the two inflammatory mediators caused by these agents was not a result of a general cytotoxic effect.

It is to be noted from the table that when taken separately, the IC₅₀ for NO inhibition of the three agents Sclareol, Naringin and Steviol are 0.04, 0.04 and 0.02, respectively. Furthermore, when used in combination with each other, said combination is even more potent, with an IC₅₀ for NO inhibition of 0.004 in the absence of Rhizobium species—, and 0.001 in the presence of Rhizobium species—. If these results are compared with the comparable IC₅₀ values for NO inhibition published for 44 selected plant extracts in the aforementioned paper by A. S. Ravipati et al. (2012), it will be seen that the values for Sclareol, Naringin and Steviol are at the lower extremity of the range of values in said paper (0.03-1.49), and in one case (Steviol) even beyond the lowest extent of that range. Similarly, if the mean value for Sclareol, Naringin and Steviol is compared with that for the 44 plants reported in the paper, it may be noted that the former (0.03) is much lower than the mean extracted from said published values (0.26).

A similar conclusion may also be drawn with regard to the inhibition of TNF-α by Sclareol, Naringin and Steviol when tested separately, with IC₅₀ values of 0.08, 0.09 and 0.08, respectively (range=0.08-0.09; mean=0.083), compared with the published results for the 44 plant extracts in A. S. Ravipati et al. (2012) (range=0.07-2.5; mean—1.04).

It may thus be concluded that the three agents selected and tested in Examples 1-3, hereinabove, all have anti-inflammatory activity, and are more potent (i.e. have a lower IC₅₀) than most of a set of 44 herbal extracts, commonly used in Chinese medicine (A. S. Ravipati et al. (2012)), with respect to NO and TNF-α inhibition.

Furthermore, it is of interest to note from Table V that even in the case of less potent anti-inflammatory plant extracts (Aster tataricus and Cyperus rotundus), said extracts are also effective as activating agents for Rhizobium species with regard to anti-fungal and anti-bacterial activity (as will be shown in Example 5, hereinbelow).

### Example 10

### Inoculation of Tomato Seedlings

### Method

Tomato seedlings were inoculated with 10 cc of each test mixtures containing Rhizobium species and various combinations of activating phytochemicals. (including the bacterial and fungal cocktails described below) 10 hours after sowing.

The health of each plant was assessed 5 days following treatment, using a semi-quantitative inoculation index (0=healthy, 5=dead).

The composition and concentration of the various test mixtures, as well as the number of different activating agents used in combination are summarized in the following two tables (all concentrations are given as % v/v):

TABLE VI

| Materials | | CONCENTRATION 3 |
|---|---|---|
| | | 3 |
| 1 | Substrate: Glucose 50%* + | 30 cc |
| | cocktail of funguses** and | |
| | bacteria's*** | |
| 2 | Rhizobium complex | 3% |
| 3 | Sclareol 98% | 0.12% |
| 4 | Naringin 98% | 0.12% |
| 5 | Nootkatone 98% | 0.12% |
| 6 | stevia | 0.01% |
| 7 | Hemp Oil contain CBD 15% | 0.00% |
| | calculated as 100% CBD | |
| 8 | Aster tataricus | 0.80% |
| 9 | Cyperus rotundus | 0.80% |
| 10 | Platycodon grandiflorus | 0.80% |
| 11 | Pleione bulbocadioides | 0.80% |
| 12 | MCT | 2.20% |
| | surfactants oil soluble | 0.50% |
| | Surfactants water soluble | 0.95% |
| | Glycerol | 9.00% |

*The Glucose 50% was mixed with water W/W
**The fungus cocktail was made of: Botrytis cinerea, Rhizoctonia solani, Pythium spp and non-pathogenic funguses used for fermentation of tomatoes.
***The bacterial cocktail was made of: Clavibacter michiganensis, Xanthomonas campestris, Pseudomonas syringae and non-pathogenic bacteria used for fermentation of tomatoes.

TABLE VII

| Treatment | Materials (from Table VI) |
|---|---|
| 1 | 1 |
| 2 | 1 + 2 − 3 |
| 3 | 1 + 2 − 3 + 3 |
| 4 | 1 + 2 − 3 + 4 − 3 |
| 5 | 1 + 2 − 3 + 5 − 3 |
| 6 | 1 + 2 − 3 + 6 − 3 |
| 7 | 1 + 2 − 3 + 7 − 3 |
| 8 | 1 + 2 − 3 + 7 − 3 + 8 − 3 |
| 9 | 1 + 2 − 3 + 7 − 3 + 9 − 3 |
| 10 | 1 + 2 − 3 + 8 − 3 |
| 11 | 1 + 2 − 3 + 9 − 3 |
| 12 | 1 + 2 − 3 + 7 − 3 + 8 − 3 + 9 − 3 |
| 13 | 1 + 2 − 3 + 3 − 3 + 4 − 3 + 5 − 4 + 6 − |
| | 4 + 7 − 3 + 8 − 3 + 9 − 3 |
| 14 | 1 + 2 − 3 + 10 − 3 |
| 15 | 1 + 2 − 3 + 11 − 3 |
| 16 | 1 + 2 − 3 + 3 − 3 + 4 − 3 + 5 − 4 + 6 − 4 + |
| | 7 − 3 + 8 − 3 + 9 − 3 + 10 − 3 + 11 − 3 |

### Results

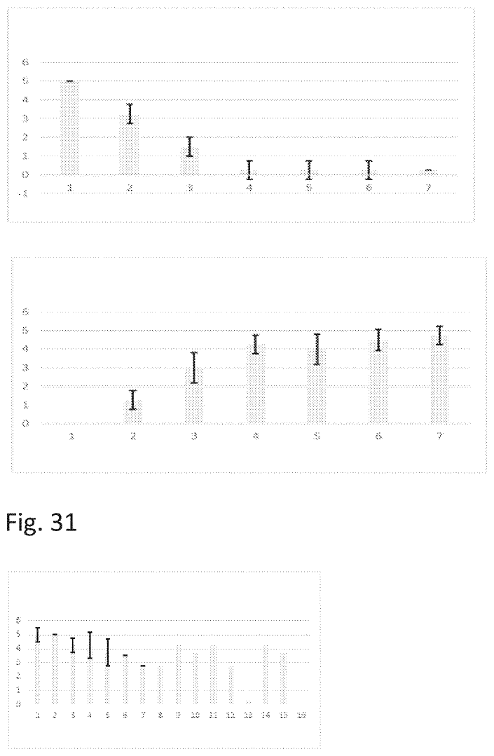

The results of this inoculation study are summarized graphically in FIG. 31. It may be seen from this figure that only test mixture 13 caused near-maximal protection of the tomato plants. As defined in Tables VI and VII, above, this treatment contained a mixture of the Rhizobium complex together with the following activating agents: sclareol, naringin, nootkatone, stevia, Hemp oil, Aster tataricus extract and Cyperus rotundus extract. The next most active treatments were 7 (Rhizobium and Hemp Oil), 8 (Rhizobium, Hemp Oil and Aster tataricus extract) and 12 (Rhizobium, Hemp Oil Aster tataricus extract and Cyperus rotundus extract).

These results indicate that the common ingredient found in the most active treatment mixtures is Cannabidiol (CBD; hemp oil), which was highly active even when present as the sole activating agent.

### Example 11

### Inoculation of Cucumber Seedlings

### Method

10 cc of each mixture of activating agents, Rhizobium species and additional components (as described in Tables I and II of Example 6, hereinabove) was sampled from the relevant petri dish and injected into 4 replicates of germinating cucumber seeds 10 hour after sowing.

The health of each plant was assessed 5 days following treatment, using a semi-quantitative inoculation index (0=healthy, 5=dead).

### Results

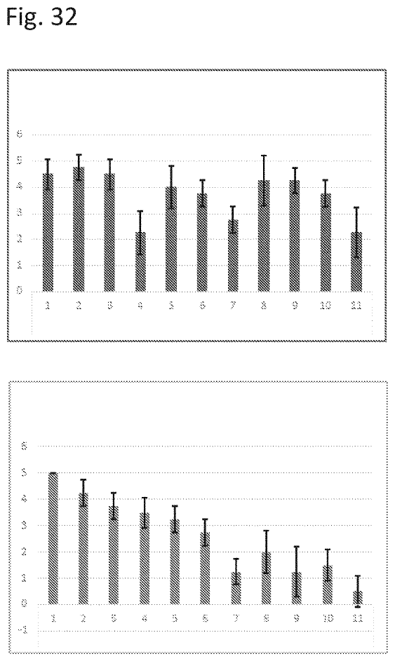

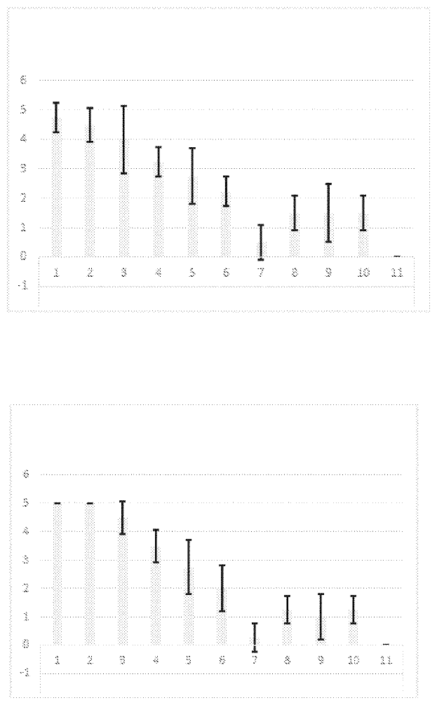

The results of this study are shown graphically in FIG. 32, in which the four separate graphs summarize the data obtained using the activating agents at concentrations 1, 2, 3 and 4 (from above to below).

As may be seen from the first (upper) graph in FIG. 32, most of the treatment protocols, when used at the lowest concentration (concentration 1) were either incapable of protecting the plants from microbial infection (inoculation index close to 5) or had minimal protective effect.

The second graph in FIG. 32 indicates that at the next higher concentration in the series (concentration 2), activation agent mixtures 6 to 11 all provide high level protection for the cucumber plants from fungal and bacterial infection. A similar result was also seen when the agents were used at concentration 3, as shown in the third graph in FIG. 32.

At the highest concentration (concentration 4; last graph in FIG. 32), the greatest protective effect is seen with activation mixtures 5 to 11.

In summary: all of the multiple-component activating agent mixtures, as well as some of the mixtures containing only one activating agent, were effective at protecting cucumber plants in vivo, when used at concentrations 2 to 4. The semi-quantitative data obtained in this study correlate very well with the appearance of the plants that were subjected to the various treatments.

### Example 12

### Effect of Compositions of the Present Invention on the Bacterial Plant Pathogen Clavibacter michiganensis sp. Michiganensis (Cmm) (1)

In this study, the effect of various combinations of Rhizobium species with activating agents on the survival of the pathogenic bacteria Clavibacter michiganensis sp. Michiganensis (Cmm) was investigated in vitro.

### Methods

Various combinations of a 3% Rhizobium sp. preparation together with an emulsion containing 5 activating agents (E-91) or one of the components of said emulsion (naringin) and a culture of the plant pathogen Cmm (10⁵-10⁶ CFU/ml final concentration) were incubated in test tubes for up to 3 days (4 replicates per combination). At the end of the 3 day incubation period the contents of the test tubes containing all these components were plated on to growth medium and the numbers of colonies of Cmm and Rhizobium species for each test condition (CFU/ml) were measured.

The emulsion containing the 5 activating agents (Sclaerol, Naringin, Nootkatone, Stevia and CBD; referred to in the results table hereinbelow as 5% plant emulsion E-91) was prepared as described in Example 6, hereinabove.

### Results

The results obtained (CFU/ml) are presented in the following table:

| | | Contact time: 3 days | |
|---|---|---|---|
| Treatment | Replicate | Cmm | Rhizobium sp. |
| Cmm | 1 | 4.6 × 10⁵ | — |
| | 2 | 5.3 × 10⁵ | — |
| | 3 | 5 × 10⁵ | — |
| | 4 | 6.1 × 10⁵ | — |
| | MEAN | 5.25 × 10⁵ | — |
| Rhizobium sp. | 1 | — | 10⁶ |
| | 2 | — | 10⁶ |
| | 3 | — | 10⁶ |
| | 4 | — | 10⁶ |
| | MEAN | — | 10⁶ |
| Cmm + | 1 | 1.7 × 10⁶ | — |
| 5% plant emulsion | 2 | 1.4 × 10⁶ | — |
| E-91 | 3 | 1.4 × 10⁶ | — |
| | 4 | 1.6 × 10⁶ | — |
| | MEAN | 1.53 × 10⁶ | — |
| Cmm + | 1 | 2 × 10² | 10⁶ |
| 5% plant emulsion | 2 | <100 | 10⁶ |
| E-91 + | 3 | 3 × 10² | 10⁶ |
| 3% Rhizobium sp. | 4 | 9 × 10¹ | 10⁶ |
| | MEAN | 172.5 | 10⁶ |
| Cmm + | 1 | 6.5 × 10⁵ | — |
| 0.1% Naringin | 2 | 8.3 × 10⁵ | — |
| | 3 | 7.7 × 10⁵ | — |
| | 4 | 8.6 × 10⁵ | — |
| | MEAN | 7.75 × 10⁵ | — |
| Cmm + | 1 | 5 × 10⁴ | 10⁶ |
| 3% Rhizobium sp. | 2 | 1.5 × 10⁵ | 10⁶ |
| | 3 | 6.3 × 10⁴ | 10⁶ |
| | 4 | 1 × 10⁵ | 10⁶ |
| | MEAN | 9.0 × 10⁴ | 10⁶ |
| Cmm + | 1 | 6.5 × 10⁵ | 10⁶ |
| 3% Rhizobium sp.+ | 2 | 3 × 10⁵ | 10⁶ |
| 0.1% Naringin | 3 | 2 × 10⁵ | 10⁶ |
| | 4 | 3 × 10⁵ | 10⁶ |
| | MEAN | 3.63 × 10⁵ | 10⁶ |

It may be seen from these results that the only test mixture which was capable of reducing the Cmm count was the combination of 5% plant emulsion E-91 and 3% Rhizobium sp. This treatment caused a massive reduction in the Cmm count, from a control value of 5.25×10⁵ to a final count of 172.5.

The combination of Naringin (as the sole activating agent) and 3% Rhizobium had no effect on the Cmm count (3.63×10⁵). It may therefore be concluded that a combination of Rhizobium and naringin alone (i.e. in the absence of any other activating or anti-inflammatory agents) is unable to kill the Cmm pathogens.

### Example 13

### Effect of Compositions of the Present Invention on the Bacterial Plant Pathogen Clavibacter michiganensis sp. Michiganensis (Cmm) (2)

### Method

This study was conducted in essentially the same manner as the study presented in Example 12. In the present study, however, the effect of the 5-component activating agent emulsion (5% plant emulsion E91) is compared with the following combinations of activating agents:

| Code | Activating agents present |
|---|---|
| 3 + 4 | Sclareol + Naringin |
| 3 + 4 + 5 | Sclareol + Naringin + Nootkatone |
| 3 + 4 + 5 + 6 | Sclareol + Naringin + Nootkatone + stevia |

### Results

The results of these comparisons are set out in the following table:

| | | Contact time: 3 days | |
|---|---|---|---|
| Treatment | Replicate | Cmm | Rhizobium sp. |
| 5% plant emulsion | 1 | 6.3 × 10⁶ | — |
| 5% plant emulsion | 2 | 5.8 × 10⁶ | — |
| E-91 + Cmm | 3 | 7.4 × 10⁶ | — |
| | MEAN | 6.5 × 10⁶ | |
| 5% plant emulsion | 1 | 9.3 × 10³ | >10⁴ |
| 3 + 4 + Cmm + | 2 | 4.4 × 10⁵ | >10⁴ |
| 3% Rhizobium sp. | 3 | 9.4 × 10⁵ | >10⁴ |
| | MEAN | 4.63 × 10⁵ | |
| 5% plant emulsion | 1 | 6.7 × 10⁵ | — |
| 3 + 4 + Cmm | 2 | 5.5 × 10⁶ | — |
| | 3 | 9.6 × 10⁴ | — |
| | MEAN | 3.4 × 10⁶ | |
| 5% plant emulsion | 1 | 7.5 × 10⁶ | >10⁴ |
| 3 + 4 + 5 + Cmm + | 2 | 5.8 × 10⁴ | >10⁴ |
| 3% Rhizobium sp. | 3 | 7.7 × 10⁵ | >10⁴ |
| | MEAN | 2.78 × 10⁶ | |
| 5% plant emulsion | 1 | 9.1 × 10⁶ | — |
| 3 + 4 + 5 + Cmm | 2 | 9.7 × 10⁶ | — |
| | 3 | 8.1 × 10⁶ | — |
| | MEAN | 8.97 × 10⁶ | |
| 5% plant emulsion | 1 | 5.2 × 10⁶ | >10⁴ |
| 3 + 4 + 5 + 6 + Cmm + | 2 | 7 × 10⁵ | >10⁴ |
| 3% Rhizobium sp. | 3 | 5.8 × 10⁵ | >10⁴ |
| | MEAN | 2.16 × 10⁶ | |
| 5% plant emulsion | 1 | 5.1 × 10⁶ | — |
| 3 + 4 + 5 + 6 + Cmm | 2 | 8 × 10⁶ | — |
| | 3 | 7.8 × 10⁶ | — |
| | MEAN | 6.97 × 10⁶ | |

It may be seen from these results that the combinations of 2, 3 or 4 activating agents together with Rhizobium (in each case, in the absence of CBD) had, in some cases, a minor inhibitory effect on the Cmm count. However, all of said partial combinations were far less effective than the complete 5-component activating agent emulsion when used in combination with Rhizobium.

### Example 14

### Effect of Compositions of the Present Invention on Bacterial Plant Pathogens: Altenaria spp. and Xanthomonas euvesicatoria

### Methods

In this study, the effect of a combination of the 5-component activating agent mixture E91 with 3% Rhizobium on the survival of two other plant pathogens—fungal species of the genus Alternaria and the gram negative bacteria Xanthomonas euvesicatoria was investigated. All materials and methods are as described hereinabove in Examples 12 and 13, except for the co-incubation time, which in this study was 2 days.

### Results

The results of this study are shown in the following table:

| | | Contact time: 2 days | |
|---|---|---|---|
| Treatment | Replicate | Rhizobium/XV | Rhizobium sp. |
| 5% plant emulsion | 1 | 1.6 × 10⁷ | >10⁴ |
| E-91 + XV + | 2 | 9 × 10⁶ | >10⁴ |
| 3% Rhizobium sp. | 3 | 8.6 × 10⁶ | >10⁴ |
| | MEAN | 1.12 × 10⁷ | |
| 5% plant emulsion | 1 | 1.7 × 10⁷ | — |
| E-91 + XV | 2 | 5.2 × 10⁷ | — |
| | 3 | 6 × 10⁷ | — |
| | MEAN | 4.3 × 10⁷ | |
| 5% plant emulsion | 1 | 20 | >10⁴ |
| E-91 + Rhizobium + | 2 | 20 | >10⁴ |
| 3% Rhizobium sp. | 3 | 100 | >10⁴ |
| | MEAN | 47 | |
| 5% plant emulsion | 1 | 1 × 10³ | — |
| E-91 + Rhizobium | 2 | 8 × 10² | — |
| | 3 | 1.1 × 10³ | — |
| | MEAN | 960 | |

### XV=Xanthomonas euvesicatoria

It may be seen from these results that the combination of the activating agents with Rhizobium caused a moderate reduction in the Xanthomonas euvesicatoria count after 2 days, as compared with the samples treated with the activating agents alone.

In the case of the Alternaria species, the reduction in the microbial count caused by the combination of the activating agents and Rhizobium as compared with the activating agents alone was much more significant.

It may be concluded that the compositions of the present invention have antimicrobial activity on a range of different bacterial and fungal species, including those species which are important plant pathogens.

1. A method for supplying the nitrogen requirements of a plant comprising administering to said plant a combination of non-pathogenic, atmospheric nitrogen-fixing bacteria and one or more activating agents.

2. The method according to claim 1, wherein the non-pathogenic, atmospheric nitrogen-fixing bacteria are members of the Rhizobium genus.

3. The method according to claim 2, wherein the bacteria are of the species Rhizobium leguminosarum.

4. The method according to claim 1, wherein the one or more activating agents are substances having anti-inflammatory activity.

5. The method according to claim 4, wherein the activating agents each have an IC₅₀ for the inhibition of NO production of less than 1.6 mg/ml and/or an IC₅₀ for the inhibition of TNF-α production of less than 2.4 mg/ml.

6. The method according to claim 1, wherein the activating agents are selected from the group consisting of: Sclareol, Naringin, Nootkatone, Steviol glycoside and cannabidiol and combinations thereof.

7. The method according to claim 6, wherein the cannabidiol is present in hemp oil.

8. The method according to claim 1, wherein the activating agent comprises cannabidiol, and optionally further comprises activating agents each having an IC₅₀ for the inhibition of NO production of less than 1.6 mg/ml and/or an IC₅₀ for the inhibition of TNF-α production of less than 2.4 mg/ml.

9. The method according to claim 1, wherein the activating agents are selected from the group consisting of extracts or other material obtained from Aster tataricus, Cyperus rotundus and combinations thereof.

10. The method according to claim 1, wherein the plant is a member of a species which is normally unable to obtain its nitrogen requirements by bacterial fixation of atmospheric nitrogen.

11. The method according to claim 10, wherein the plant species is a member of the Graminaea family.

12. The method according to claim 11, wherein the plant species is maize.

13. The method according to claim 11, wherein the plant species is wheat.

14. The method according to claim 1, further comprising the administration of one or more phosphorous-containing fertilizers.

15. The method according to claim 14, wherein the fertilizer is Calirus.

16. The method according to claim 1, wherein the combination of non-pathogenic, atmospheric nitrogen-fixing bacteria and one or more activating agents are administered by means selected from the group consisting of: application of slow-release granules to the soil in which the plants are being grown, seed coating and spraying the sowing trench or furrow.

17. The method according to claim 1, wherein the non-pathogenic, atmospheric nitrogen-fixing bacteria and the one or more activating agents are administered together in a single composition.

18. The method according to claim 1, wherein the non-pathogenic, atmospheric nitrogen-fixing bacteria and the one or more activating agents are administered in separate compositions.

19. A composition comprising a mixture of non-pathogenic nitrogen-fixing bacteria and one or more activating agents.

20. The composition according to claim 19, wherein the non-pathogenic, atmospheric nitrogen-fixing bacteria are members of the Rhizobium genus.

21. The composition according to claim 20, wherein the bacteria are of the species Rhizobium leguminosarum.

22. The composition according to claim 19, wherein the one or more activating agents are substances having anti-inflammatory activity.

23. The composition according to claim 22, wherein the activating agents each have an IC₅₀ for the inhibition of NO production of less than 1.6 mg/ml and/or an IC₅₀ for the inhibition of TNF-α production of less than 2.4 mg/ml.

24. The composition according to claim 22, wherein the activating agents are selected from the group consisting of: Sclareol, Naringin, Nootkatone, Steviol glycoside and cannabidiol and combinations thereof.

25. The composition according to claim 22, wherein the activating agents comprise cannabidiol.

26. The composition according to claim 22, wherein the activating agents comprise cannabidiol and optionally further comprise activating agents each having an IC₅₀ for the inhibition of NO production of less than 1.6 mg/ml and/or an IC₅₀ for the inhibition of TNF-α a production of less than 2.4 mg/ml.

27. The composition according to claim 22, wherein the activating agents are selected from the group consisting of: extracts or other material obtained from Aster tataricus, Cyperus rotundus and combinations thereof.

28. The composition according to claim 19, further comprising one or more phosphorous-containing fertilizers.

29. The composition according to claim 28, wherein the phosphorous-containing fertilizer is Calirus.

30. A method for increasing the yield of a plant of agricultural or horticultural importance by means of:
a) providing a composition according to any one of claims 19-29; and
b) administering the composition of step (a) to said host species.

31. A method for increasing the yield of a plant of agricultural or horticultural importance by means of:
a) providing separately:
(i) a composition comprising one or more nitrogen fixing non-pathogenic bacteria; and
(ii) a composition comprising one or more activating agents as defined in any one of claims 23-27; and
b) separately administering each of compositions (i) and (ii) to said host species.

32. The method according to claim 31, wherein the nitrogen fixing non-pathogenic bacteria are members of the Rhizobium genus.

33. The method according to claim 32, wherein the bacteria are of the species Rhizobium leguminosarum.

34. The method according to claim 30 or claim 31, wherein the plant of agricultural or horticultural importance is a member of a species which is normally unable to obtain its nitrogen requirements by bacterial fixation of atmospheric nitrogen.

35. The method according to claim 34, wherein the plant species is a member of the Graminaea family.

36. The method according to claim 35, wherein the plant species is maize.

37. The method according to claim 35, wherein the plant species is wheat.
